(19) United States
(12) Patent Application Publication
(10) Pub. No.: US 2026/0134540 A1
(43) Pub. Date: May 14, 2026

(54) METHODS AND APPARATUSES FOR AUTOMATING ANALYSIS OF NEAR-INFRARED INTRAORAL SCANS

(71) Applicant: Align Technology, Inc., San Jose, CA (US)

(72) Inventors: Ido TISHEL, Kfar Bilu (IL); Roie COHEN, Givatayim (IL); Hila SEGEV, Ramat Ishay (IL); Uriel BARRON, Tel Aviv (IL)

(21) Appl. No.: 19/386,213

(22) Filed: Nov. 11, 2025

Related U.S. Application Data
(60) Provisional application No. 63/719,093, filed on Nov. 11, 2024.
(60) Provisional application No. 63/781,300, filed on Mar. 31, 2025.

(51) Int. Cl. G06T 7/00; A61B 1/24; A61B 5/00; A61C 9/00; A61C 13/34; G06T 7/10; G06V 10/25; G06V 10/26; G06V 10/764; G06V 10/82

(52) U.S. Cl. CPC G06T 7/0014; A61B 1/24; A61B 5/4547; A61C 9/0053; A61C 13/34; G06T 7/10; G06V 10/25; G06V 10/26; G06V 10/764; G06V 10/82; G06T 2200/24; G06T 2207/10024; G06T 2207/10048; G06T 2207/20084; G06T 2207/20104; G06T 2207/30036; G06V 2201/03

(57) ABSTRACT

Described herein are method and apparatuses for enhancing the display and analysis intraoral scan data, including but not limited to near-infrared scan data. Thes methods and apparatuses described herein may be used to select a subset of images (e.g., 2D images) from an intraoral scan efficiently and accurately by determining which scans are most likely to include information of relevance including information about one or more defects or flaws in the patient's dentition. These methods and apparatuses may also display the selected subset of the associated 2D images in a determined sequence.

### CLAIM OF PRIORITY

This patent application claims priority to U.S. Provisional Ser. No. 63/719,093, titled “REVIEW TOOL VIDEO CLIP,” filed on Nov. 11, 2024, and U.S. Provisional Ser. No. 63/781,300, titled “REVIEW TOOL VIDEO CLIP,” filed on Mar. 31, 2025, each of which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The present disclosure relates generally to three-dimensional imaging, and more particularly to intraoral three-dimensional imaging.

### BACKGROUND

Digital dental impressions utilize intraoral scanning to generate three-dimensional digital models of an intraoral three-dimensional surface of a subject. Digital intraoral scanners may use structured light or a combination of structured light projectors and cameras disposed within the intraoral scanner for three-dimensional imaging.

US 2019/0388193 to Saphier et al., which is assigned to the assignee of the present application and is incorporated herein by reference, describes an apparatus for intraoral scanning including an elongate handheld wand that has a probe. One or more light projectors and two or more cameras are disposed within the probe. The light projectors each have a pattern generating optical element, which may use diffraction or refraction to form a light pattern. Each camera may be configured to focus between 1 mm and 30 mm from a lens that is farthest from the camera sensor. Other applications are also described.

US 2019/0388194 to Atiya et al., which is assigned to the assignee of the present application and is incorporated herein by reference, describes a handheld wand including a probe at a distal end of the elongate handheld wand. The probe includes a light projector and a light field camera. The light projector includes a light source and a pattern generator configured to generate a light pattern. The light field camera includes a light field camera sensor. The light field camera sensor includes (a) an image sensor including an array of sensor pixels and (b) an array of micro-lenses disposed in front of the image sensor such that each micro-lens is disposed over a sub-array of the array of sensor pixels. Other applications are also described.

US 2020/0404243 to Saphier et al., which is assigned to the assignee of the present application and is incorporated herein by reference, describes a method for generating a 3D image, including driving structured light projector(s) to project a pattern of light on an intraoral 3D surface, and driving camera(s) to capture images, each image including at least a portion of the projected pattern, each one of the camera(s) comprising an array of pixels. A processor compares a series of images captured by each camera and determines which of the portions of the projected pattern can be tracked across the images. The processor constructs a three-dimensional model of the intraoral three-dimensional surface based at least in part on the comparison of the series of images. Other embodiments are also described.

PCT Publication WO 2018/152374 to Ozerov et al., which is assigned to the assignee of the present application and is incorporated herein by reference, describes processing logic which makes a comparison between first image data and second image data of a dental arch and determines a plurality of spatial differences between a first representation of the dental arch in the first image data and a second representation of the dental arch in the second image data. The processing logic determines that a first spatial difference is attributable to scanner inaccuracy and that a second spatial difference is attributable to a clinical change to the dental arch. The processing logic generates a third representation of the dental arch that is a modified version of the second representation, wherein the first spatial difference is removed in the third representation, and wherein the third representation comprises a visual enhancement that accentuates the second spatial difference. Other embodiments are also described.

US20240307158 to Levy et al., which is assigned to the assignee of the present application and is incorporated herein by reference, describes techniques for selecting images from a plurality of images generated by an intraoral scanner. A method includes receiving a plurality of images of a dental site generated by an intraoral scanner, identifying a subset of images from the plurality of images that satisfy one or more selection criteria, selecting the subset of images that satisfy the one or more selection criteria, and discarding or ignoring a remainder of images of the plurality of images that are not included in the subset of images. Other embodiments are also described.

US20210068773 to Moshe et al., which is assigned to the same assignee of the present application and which is incorporated herein by reference, describes devices and methods for generating a panoramic rendering of a subject's teeth. Methods and processes are provided to image the subject's teeth with a dental scan. Methods and processes are also provided to automatically 3D render the subject's teeth with the scan images. Methods and apparatuses are also provided to generate simulated panoramic views of the subject's dentition from various perspectives. Other embodiments are also described.

All of the methods and apparatuses described herein, in any combination, are herein contemplated and can be used to achieve the benefits as described herein.

### SUMMARY OF THE DISCLOSURE

For some applications of the present invention, an intraoral scanner generates a plurality of scans of an intraoral three-dimensional (3D) surface of an intraoral object during intraoral scanning. Additionally, the intraoral scanner generates two-dimensional near-infrared (NIR) images of the intraoral object and/or 2D color images of the intraoral 3D surface during the intraoral scanning. When considering an area of a 3D model based on the intraoral scans, e.g., in order to identify a dental pathology, e.g., caries, cracks, wear, etc., it may be useful for a dentist or dental technician to view some of the 2D NIR and/or 2D color images corresponding to that area of the 3D model that were captured during the intraoral scanning from varying viewing angles and/or, in the case of NIR, under varying NIR-illumination angles. The inventors have realized that instead of the dentist or dental technician manipulating a view of the 3D model in order to look for a good viewing angle for the pathology identification, it would be advantageous for the dentist or dental technician to be automatically shown a series of the 2D NIR and/or 2D color images selected by a computer processor of the intraoral scanning system that correspond to an area of the 3D model that the dentist or dental technician wishes to consider.

For example, the dentist or dental technician may wish to consider one or more interproximal areas of a dental arch of a subject when looking for caries. Thus, for some applications, the computer processor selects a series of the 2D NIR and/or 2D color images to display that were generated during the intraoral scanning of the one or more interproximal areas from various viewing angles and/or, in the case of NIR, utilizing various NIR-illumination angles. This allows the dentist or dental technician to consider the one or more interproximal areas easily without having to manipulate the 3D model on the display.

For some applications, the series of selected images may be displayed in a sequence determined by the computer processor. For example, when the series of images selected by the computer processor vary in viewing angle, the computer processor may display the images in a sequence based on the viewing angle of each image such that the dentist or dental technician sees the specific area of the 3D model from different viewing angles, as if seeing a video clip. Alternatively, for some applications, the series of images selected by the computer processor may share similar viewing angles and, instead of displaying the images sequentially, the computer processor may merge the images into a merged image corresponding to the area of the 3D model. This may be advantageous, for example, in order to increase image resolution or increase a signal to noise ratio in the displayed images.

For example, described herein are intraoral scanning systems, the system comprising: an intraoral scanner configured to: generate a plurality of scans of an intraoral three-dimensional (3D) surface of an intraoral object during intraoral scanning of the intraoral 3D surface, and generate two-dimensional (2D) near-infrared (NIR) images of the intraoral object during intraoral scanning of the intraoral 3D surface, and at least one computer processor configured to: receive the plurality of scans, build a 3D model of the intraoral 3D surface based on the plurality scans, associate at least a portion of the 2D NIR images with the 3D model such that each of the associated 2D NIR images corresponds to a position and viewing angle relative to the 3D model, display to a user the 3D model of the intraoral 3D surface, select a subset of the associated 2D NIR images that correspond to an area of the 3D model, determine a sequence for displaying the selected subset of the associated 2D NIR images, and display the selected subset of the associated 2D NIR images in the determined sequence. The at least one computer processor may be configured to display the selected subset by projecting the selected subset of the associated 2D NIR images onto the 3D model. The at least one computer processor may be configured to facilitate observation of caries by selecting the subset of the associated 2D NIR images based on respective viewing angles relative to the intraoral 3D surface of the associated 2D NIR images or respective NIR-illumination angles relative to the intraoral 3D surface of the associated 2D NIR images. The at least one computer processor may be configured to automatically identify caries in the intraoral object based on the generated 2D NIR images. The at least one computer processor may be configured to automatically select the area of the 3D model based on the identification of caries in the intraoral object.

The at least one computer processor may be configured to select the subset of the associated 2D NIR images based on the identification of caries in the intraoral object. The at least one computer processor may be further configured to normalize the subset of the associated 2D NIR images with respect to each other. The at least one computer processor may be configured to normalize the subset of the associated 2D NIR images by normalizing a brightness of at least one 2D NIR image of the subset with respect to a brightness of at least one other 2D NIR image of the subset. The at least one computer processor may be configured to normalize the subset of the associated 2D NIR images by normalizing a contrast within at least one 2D NIR image of the subset with respect to a contrast within at least one other 2D NIR image of the subset.

The at least one computer processor may be further configured to identify a common region of interest as seen in each image of the selected subset of the associated 2D NIR images, and wherein the at least one computer processor may be further configured to display the selected subset in the determined sequence while maintaining the identified common region of interest spatially stable during transitioning by the computer processor from one 2D NIR image to the next within the selected subset. The at least one computer processor maintains the identified common region of interest spatially stable during transitioning by aligning the selected subset of the associated 2D NIR images with respect to each other. The at least one computer processor maintains the identified common region of interest spatially stable during transitioning by morphing at least one image of the selected subset of the associated 2D NIR images to the next. The at least one computer processor maintains the identified common region of interest spatially stable during transitioning by cropping at least one image of the selected subset of the associated 2D NIR images. The intraoral scanning system may include at least one computer processor may be configured to display the selected subset of the plurality of 2D NIR images in the determined sequence on a display screen, and the at least one computer processor maintains the identified common region of interest spatially stable during transitioning by displaying at least a first image of the selected subset centered around a first point on the display screen and displaying at least a second image of the selected subset centered around a second point, different from the first point, on the display screen.

The at least one computer processor maintains the identified common region of interest spatially stable during transitioning by rotating at least one image of the selected subset of the associated 2D NIR images. The area of the 3D model corresponds to an interproximal area of the intraoral 3D surface. The at least one computer processor may be further configured to:
automatically identify one or more interproximal areas of the intraoral 3D surface, each identified interproximal area corresponding respectively to an area of the 3D model; for each identified interproximal area, automatically select a subset of the associated 2D NIR images that correspond to the respective area of the 3D model; and for each selected subset: determine a sequence for displaying the selected subset of the associated 2D NIR images, and display the selected subset of the associated 2D NIR images in the determined sequence.

The at least one computer processor may be further configured to: receive user input associated with a given area of the 3D model of the intraoral 3D surface, and in response to receiving the user input, select a subset of the associated 2D NIR images that correspond to the given area of the 3D model. The given area may be an interproximal area on the 3D model corresponding to an interproximal area on the intraoral 3D surface, and the at least one computer processor may be configured to receive the user input in response to the user selecting a particular one or more interproximal areas on the 3D model. The at least one computer processor may be configured to receive the user input by the user adjusting an indicator on a view of the 3D model, wherein the given area may be an area on the 3D model that may be associated with the indicator. The indicator comprises an area-selecting indicator, wherein the user input comprises the user moving the area-selecting indicator, and wherein the given area may be the area of the 3D model that may be within the area-selecting indicator.

For one or more given viewing angles from which no 2D NIR images were generated during the intraoral scanning, the at least one computer processor may be further configured to synthesize one or more synthesized 2D NIR images of the intraoral 3D surface based on 2D NIR images generated during the intraoral scanning from viewing angles nearby the one or more given viewing angles, and the computer processor may be configured to display the selected subset of the associated 2D NIR images in the determined sequence by augmenting the selected subset of the associated 2D NIR images with the one or more synthesized 2D NIR images corresponding respectively to one or more viewing angles for which there may be no associated 2D NIR image.

Also described herein are intraoral scanning systems including: an intraoral scanner configured to: generate a plurality scans of an intraoral three-dimensional (3D) surface of an intraoral object during intraoral scanning of the intraoral 3D surface, and generate two-dimensional (2D) color images of the intraoral 3D surface, during intraoral scanning of the intraoral 3D surface; and at least one computer processor configured to: receive the plurality of scans, build a 3D model of the intraoral 3D surface based on the plurality of scans, associate at least a portion of the 2D color images with the 3D model such that each of the associated 2D color images corresponds to a position and viewing angle relative to the 3D model, display to a user the 3D model of the intraoral 3D surface, select a subset of the associated 2D color images that correspond to an area of the 3D model, determine a sequence for displaying the selected subset of the associated 2D color images, and display the selected subset of the associated 2D color images in the determined sequence. The at least one computer processor may be configured to display the selected subset by displaying the selected subset of the associated 2D color images projected onto the 3D model. The 2D color images may be images captured under white light illumination. The 2D color images may be fluorescence images. The fluorescence images may be images of the intraoral 3D surface fluorescing in response to ultraviolet (UV) illumination. The fluorescence images may be images of the intraoral 3D surface fluorescing in response to red light illumination. The at least one computer processor may be configured to facilitate observation of a dental pathology by selecting the subset of the associated 2D color images based on respective viewing angles relative to the intraoral 3D surface of the associated 2D color images. The dental pathology may be caries.

The at least one computer processor may be further configured to automatically identify a dental pathology based on the generated 2D color images. The at least one computer processor may be configured to automatically select the area of the 3D model based on the identification of the dental pathology. The at least one computer processor may be configured to select the subset of the associated 2D color images based on the identification of the dental pathology. The dental pathology may be caries. The at least one computer processor may be configured to automatically select the area of the 3D model based on the identification of caries in the intraoral object. The at least one computer processor may be configured to select the subset of the associated 2D color images based on the identification of caries in the intraoral object The at least one computer processor may be further configured to normalize the subset of the associated 2D color images with respect to each other. The at least one computer processor may be configured to normalize the subset of the associated 2D color images by normalizing a brightness of at least one 2D color image of the subset with respect to a brightness of at least one other 2D color image of the subset. The at least one computer processor may be configured to normalize the subset of the associated 2D color images by normalizing a contrast within at least one 2D color image of the subset with respect to a contrast within at least one other 2D color image of the subset.

The at least one computer processor may be further configured to identify a common region of interest as seen in each image of the selected subset of the associated 2D color images, and wherein the at least one computer processor may be further configured to display the selected subset in the determined sequence while maintaining the identified common region of interest spatially stable during transitioning by the computer processor from one 2D color image to the next within the selected subset. The at least one computer processor maintains the identified common region of interest spatially stable during transitioning by aligning the selected subset of the associated 2D color images with respect to each other. The at least one computer processor maintains the identified common region of interest spatially stable during transitioning by morphing at least one image of the selected subset of the associated 2D color images to at least one other image of the selected subset of the associated 2D color images the next The at least one computer processor maintains the identified common region of interest spatially stable during transitioning by cropping at least one image of the selected subset of the associated 2D color images. The at least one computer processor may be configured to display the selected subset of the plurality of 2D color images in the determined sequence on a display screen, and the at least one computer processor maintains the identified common region of interest spatially stable during transitioning by displaying at least a first image of the selected subset centered around a first point on the display screen and displaying at least a second image of the selected subset centered around a second point, different from the first point, on the display screen. The at least one computer processor maintains the identified common region of interest spatially stable during transitioning by rotating at least one image of the selected subset of the associated 2D color images.

The area of the 3D model corresponds to an interproximal area of the intraoral 3D surface. The at least one computer processor may be further configured to: automatically identify one or more interproximal areas of the intraoral 3D surface, each identified interproximal area corresponding respectively to an area of the 3D model; for each identified interproximal area, automatically select a subset of the associated 2D color images that correspond to the respective area of the 3D model; and for each selected subset: determine a sequence for displaying the selected subset of the associated 2D color images, and display the selected subset of the associated 2D color images in the determined sequence. The at least one computer processor may be further configured to: receive user input associated with a given area of the 3D model of the intraoral 3D surface, and in response to receiving the user input, select the subset of the associated 2D color images that correspond to the given area of the 3D model.

The given area may be an interproximal area on the 3D model corresponding to an interproximal area on the intraoral 3D surface, and the at least one computer processor may be configured to receive the user input in response to the user selecting a particular one or more interproximal areas on the 3D model. The at least one computer processor may be configured to receive the user input by the user adjusting an indicator on a view of the 3D model, wherein the given area may be an area on the 3D model that may be associated with the indicator. The indicator may comprise an area-selecting indicator, wherein the user input comprises the user moving the area-selecting indicator, and wherein the given area may be the area of the 3D model that may be within the area-selecting indicator.

In some cases, for one or more given viewing angles from which no 2D color images were generated during the intraoral scanning, the at least one computer processor may be further configured to synthesize one or more synthesized 2D color images of the intraoral 3D surface based on 2D color images generated during the intraoral scanning from viewing angles nearby the one or more given viewing angles, and the computer processor may be configured to display the selected subset of the associated 2D color images in the determined sequence by augmenting the selected subset of the associated 2D color images with the one or more synthesized 2D color images corresponding respectively to one or more viewing angles for which there may be no associated 2D color image.

Also described herein are intraoral scanning system including: an intraoral scanner configured to: generate a plurality of scans of an intraoral three-dimensional (3D) surface of an intraoral object during intraoral scanning of the intraoral 3D surface, and generate two-dimensional (2D) near-infrared (NIR) images of the intraoral object during intraoral scanning of the intraoral 3D surface, and at least one computer processor configured to: receive the plurality of scans, build a 3D model of the intraoral 3D surface based on the plurality scans, associate at least a portion of the 2D NIR images with the 3D model such that each of the associated 2D NIR images corresponds to a position and viewing angle relative to the 3D model, display to a user the 3D model of the intraoral 3D surface, select a subset of the associated 2D NIR images that correspond to an area of the 3D model, merge the selected subset of the associated 2D NIR images into a merged 2D NIR image corresponding to the area, and display the merged 2D NIR image. The at least one computer processor may be configured to display the merged 2D NIR image by projecting the merged 2D NIR image onto the 3D model.

For a given area of the merged image, the at least one computer processor may be configured to select specific sub-regions from each image of the selected subset to use for the given area of the merged image. The at least one computer processor may be configured to facilitate observation of caries by selecting the subset of the associated 2D NIR images based on respective viewing angles relative to the intraoral 3D surface of the plurality of 2D NIR images or respective NIR-illumination angles relative to the intraoral 3D surface of the plurality of 2D NIR images. The at least one computer processor may be further configured to automatically identify caries in the intraoral object based on the generated 2D NIR images. The at least one computer processor may be configured to automatically select the area of the 3D model based on the identification of caries in the intraoral object. The at least one computer processor may be configured to select the subset of the associated 2D NIR images based on the identification of caries in the intraoral object. The at least one computer processor may be further configured to normalize the selected subset of the associated 2D NIR images with respect to each other and, based on the normalizing of the selected subset of the associated 2D NIR images, merge the selected subset of the associated 2D NIR images into the merged 2D NIR image.

The at least one computer processor may be configured to normalize the subset of the associated 2D NIR images by normalizing a brightness of at least one 2D NIR image of the subset with respect to a brightness of at least one other 2D NIR image of the subset. The at least one computer processor may be configured to normalize the subset of the associated 2D NIR images by normalizing a contrast within at least one 2D NIR image of the subset with respect to a contrast within at least one other 2D NIR image of the subset. The at least one computer processor may be further configured to identify a common region of interest as seen in each image of the subset of the associated 2D NIR images, and wherein the at least one computer processor may be further configured to align the common region of interest as seen in each image of the subset of the associated 2D NIR images prior to merging the selected subset of the associated 2D NIR images into the merged 2D NIR image.

Based on the aligning of the common region of interest as seen in each image of the subset of the associated 2D NIR images prior to merging, the at least one computer processor may be configured to merge the selected subset of the associated 2D NIR images by stacking the selected subset of the associated 2D NIR images into a stacked image. For a given area of the stacked image, the at least one computer processor may be configured to select specific sub-regions from each image of the selected subset to use for the given area of the stacked image. The at least one computer processor aligns the identified common region of interest as seen in each image of the subset by cropping at least one image of the selected subset of the associated 2D NIR images. The at least one computer processor aligns the identified common region of interest as seen in each image of the subset by rotating at least one image of the selected subset of the associated 2D NIR images.

The at least one computer processor aligns the identified common region of interest as seen in each image of the subset by morphing at least one image of the selected subset of the associated 2D NIR images to the next at least one other image of the selected subset of the associated 2D NIR images. The area of the 3D model corresponds to an interproximal area of the intraoral 3D surface. The at least one computer processor may be further configured to: automatically identify one or more interproximal areas of the intraoral 3D surface, each identified interproximal area corresponding respectively to an area of the 3D model; for each identified interproximal area automatically select a subset of the associated 2D NIR images that correspond to the respective area of the 3D model; and for each selected subset: merge the selected subset of the associated 2D NIR images into a merged 2D NIR image, and display the merged 2D NIR image.

The at least one computer processor may be further configured to: receive user input associated with a given area of the 3D model of the intraoral 3D surface, and in response to receiving the user input, select the subset of the associated 2D NIR images that correspond to the given area of the 3D model. The given area may be an interproximal area on the 3D model corresponding to an interproximal area on the intraoral 3D surface, and the at least one computer processor may be configured to receive the user input in response to the user selecting a particular one or more interproximal areas on the 3D model. The at least one computer processor may be configured to receive the user input by the user adjusting an indicator on a view of the 3D model, wherein the given area may be an area on the 3D model that may be associated with the indicator. The indicator comprises an area-selecting indicator, wherein the user input comprises the user moving the area-selecting indicator, and wherein the given area may be the area of the 3D model that may be within the area-selecting indicator.

In some cases the intraoral scanner may be further configured to generate two-dimensional (2D) color images of the intraoral 3D surface, during intraoral scanning of the intraoral 3D surface, and the at least one computer processor may be further configured to:
associate at least a portion of the 2D color images to with the 3D model such that each of the associated 2D color images corresponds to a position and viewing angle relative to the 3D model, select a subset of the associated 2D NIR images that correspond to an area of the 3D model, select a subset of the associated 2D color images that correspond to the same area of the 3D model, merge the selected subset of the associated 2D NIR images with the selected subset of the associated 2D color images into to create a merged 2D NIR-color image corresponding to the area of the 3D model, and display the merged 2D NIR-color image.

Also described herein are intraoral scanning systems including: (A) an intraoral scanner configured to: (i) generate a plurality scans of an intraoral three-dimensional (3D) surface of an intraoral object during intraoral scanning of the intraoral 3D surface, and (ii) generate two-dimensional (2D) color images of the intraoral 3D surface, during intraoral scanning of the intraoral 3D surface; and (B) at least one computer processor configured to: receive the plurality of scans, build a 3D model of the intraoral 3D surface based on the plurality of scans, associate at least a portion of the 2D color images with the 3D model such that each of the associated 2D color images corresponds to a position and viewing angle relative to the 3D model, display to a user the 3D model of the intraoral 3D surface, select a subset of the associated 2D color images that correspond to an area of the 3D model, merge the selected subset of the associated 2D color images into a merged 2D color image corresponding to the area, and display the merged 2D color image. The at least one computer processor may be configured to display the merged 2D color image by projecting the merged 2D color image onto the 3D model. The 2D color images may be images captured under white light illumination. The 2D color images may be fluorescence images. The fluorescence images may be images of the intraoral 3D surface fluorescing in response to ultraviolet (UV) illumination. The fluorescence images may be images of the intraoral 3D surface fluorescing in response to red light illumination.

For a given area of the merged image, the at least one computer processor may be configured to select specific sub-regions from each image of the selected subset to use for the given area of the merged image. The at least one computer processor may be configured to facilitate observation of a dental pathology by selecting the subset of the associated 2D color images based on respective viewing angles relative to the intraoral 3D surface of the associated 2D color images. The dental pathology may be caries. The at least one computer processor may be further configured to automatically identify a dental pathology based on the generated 2D color images. The at least one computer processor may be configured to automatically select the area of the 3D model based on the identification of the dental pathology. The at least one computer processor may be configured to select the subset of the associated 2D color images that based on the identification of the dental pathology. The dental pathology may be caries.

The at least one computer processor may be configured to automatically select the area of the 3D model based on the identification of caries in the intraoral object. The at least one computer processor may be configured to select the subset of the associated 2D color images that based on the identification of caries in the intraoral object. The at least one computer processor may be further configured to normalize the selected subset of the associated 2D color images with respect to each other and based on the normalizing of the selected subset of the associated 2D color images, merge the selected subset of the associated 2D color images into the merged 2D color image. The at least one computer processor may be configured to normalize the subset of the associated 2D color images by normalizing a brightness of at least one 2D color image of the subset with respect to a brightness of at least one other 2D color image of the subset. The at least one computer processor may be configured to normalize the subset of the associated 2D color images by normalizing a contrast within at least one 2D color image of the subset with respect to a contrast within at least one other 2D color image of the subset. The at least one computer processor may be further configured to identify a common region of interest as seen in each image of the subset of the associated 2D color images, and wherein the at least one computer processor may be further configured to align the common region of interest as seen in each image of the subset of the associated 2D color images prior to merging the selected subset of the associated 2D color images into the merged 2D color image.

Based on the aligning of the common region of interest as seen in each image of the subset of the associated 2D color images prior to the merging, the at least one computer processor may be configured to merge the selected subset of the associated 2D color images by stacking the selected subset of the associated 2D color images into a stacked image. For a given area of the stacked image, the at least one computer processor may be configured to select specific sub-regions from each image of the selected subset to use for the given area of the stacked image. The at least one computer processor aligns the identified common region of interest as seen in each image of the subset by cropping at least one image of the selected subset of the associated 2D color images. The at least one computer processor aligns the identified common region of interest as seen in each of image of the subset by rotating at least one image of the selected subset of the associated 2D color images. The at least one computer processor aligns the identified common region of interest as seen in each image of the subset by morphing at least one image of the selected subset of the associated 2D color images.

The area of the 3D model corresponds to an interproximal area of the intraoral 3D surface. The at least one computer processor may be further configured to: automatically identify one or more interproximal areas of the intraoral 3D surface, each identified interproximal area corresponding respectively to an area of the 3D model; for each identified interproximal area, automatically select a subset of the associated 2D color images that correspond to the respective area of the 3D model; and for each selected subset: merge the selected subset of the associated 2D color images into a merged 2D color image, and display the merged 2D color image. The at least one computer processor may be further configured to: receive user input associated with a given area of the 3D model of the intraoral 3D surface, and, in response to receiving the user input, select the subset of the associated 2D color images that correspond to the given area of the 3D model. The given area may be an interproximal area on the 3D model corresponding to an interproximal area on the intraoral 3D surface, and the at least one computer processor may be configured to receive the user input in response to the user selecting a particular one or more interproximal areas on the 3D model.

The at least one computer processor may be configured to receive the user input by the user adjusting an indicator on a view of the 3D model, wherein the given area may be an area on the 3D model that may be associated with the indicator. The indicator comprises an area-selecting indicator, wherein the user input comprises the user moving the area-selecting indicator, and wherein the given area may be the area of the 3D model that may be within the area-selecting indicator.

In general, described herein are methods and apparatuses (e.g., systems, device, including software) for reducing the number of images (e.g., image files) form an intraoral scan. Any of these methods and apparatuses may be used to form a reduced or compact coverage map for an intraoral scan. For example, described herein are intraoral scanning systems comprising: an intraoral scanner configured to generate a plurality of two-dimensional (2D) surface images and a plurality of 2D near-infrared (near-IR) images of a subject's intraoral cavity; and at least one processor configured to: generate or receive a three-dimensional (3D) model of an intraoral 3D surface based on the plurality of surface 2D images; identify, based on the 3D model, one or more regions from the plurality of 2D near-IR images each having one or more features of interest; determine a score for each of the one or more regions using a physics-based classifier and a trained machine learning agent; generate a reduced plurality of near-IR images based on the score from the plurality of 2D near-IR images; and store and/or transmit the reduced plurality of near-IR images. The at least one processor may be configured to generate the reduced plurality of near-IR images based on the score and relative position of the near-IR images on the 3D model. The at least one processor may be configured to generate the reduced plurality of near-IR images by removing or deleting images from the plurality of near-IR images to form the reduced plurality of near-IR images.

The one or more features of interest may comprise one or more of: interproximal regions, caries, cracks, wear, grinding, and/or abscesses. For example, the one or more features of interest may comprise caries; in some cases the one or more features of interest may comprise interproximal regions. The at least one processor may be further configured to segment the 3D model. Any of these systems or methods may be configured to display a subset of 2D images that correspond to a predetermined range of a user-selected area from the reduced plurality of near-IR images. For example, the at least one processor may be further configured to receive the user-selected area from a user interface displaying the 3D model. In any of these methods and apparatuses, using a physics-based classifier and the trained machine learning agent may comprise determining the score using a physics-based scene classifier and using the trained machine learning agent focusing on local regions from the 2D images for each of the one or more regions from the plurality of 2D near-IR images.

The trained machine learning agent may be a convolutional neural network (CNN). The at least one processor may be further configured to generate the reduced plurality of near-IR images by preserving a predetermined number or percentage images of near-IR images having the highest-ranking scores. The at least one processor may be further configured to normalize the scores. The at least one processor may be configured to determine the score for each of the one or more regions of each 2D near-IR image at least in part based on the physics-based classifier comprising an angle between a reference point and a camera and/or a near-IR light source corresponding to the 2D near-IR image. The trained machine learning agent may be configured to be used in combination with one or more physics-based classifiers including one or more geometric relationship between a camera, a reference point in the one or more regions, a camera refraction, a tool surface in the one or more regions, a tooth axis, a first light source, and/or a second light source. The trained machine learning agent may be configured to be used in combination with the physics-based classifier comprising one or more classifiers including one or more of: a cosine of an angle between a camera and a normal at a reference point, a Z absolute value of the reference point to a camera vector; a camera to reference point distance in mm; a cosine of an angle between a camera direction and the reference point; an absolute of z value of a camera direction vector; a cosine of the angle between a camera refraction and a tooth axis; an absolute z value of a light source direction vector; a cosine of an angle between a first light source and a normal; a cosine of an angle between the first light source direction and the reference point; a distance between a first light source location and the reference point in mm; a distance between a light source location and a camera in mm; a light source luminosity; an absolute of z value of the reference point to the light source vector; a cosine of an angle between a second light source direction and a normal; a second light source luminosity; an absolute of a z value of the reference point to a second light source vector; a cosine of an angle between the second light source direction and the reference point; a distance between the second light source location and the reference point in mm; and/or a distance between the second light source location and the camera in mm. The at least one processor may be configured to store and/or transmit the 3D model with the reduced plurality of 2D near-IR images forming the reduced plurality of near-IR images.

The present invention or inventions will be more fully understood from the following detailed description of applications thereof, taken together with the drawings.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 is a schematic illustration showing an intraoral scanning system including an intraoral scanner and a computer processor, in accordance with some applications of the present invention;

FIG. 2A shows a block diagram relating to the use of the intraoral scanning system, in accordance with some applications of the present invention;

FIG. 2B shows a flowchart illustrating steps performed by the computer processor, in accordance with some applications of the present invention;

FIGS. 3A-E depict various ways in which a common region of interest as seen in each image of a selected subset of images is maintained spatially stable during transition by the computer processor from one image to the next within the selected subset, in accordance with some applications of the present invention;

FIGS. 4A-B are schematic illustrations showing a 3D model of a dental arch of a subject, in accordance with some applications of the present invention.

FIG. 4C shows a 3D model of a dental arch from scans generated during the intraoral scanning associated at least a portion of 2D NIR images.

FIG. 5 shows a flowchart illustrating steps performed by the computer processor, in accordance with some applications of the present invention.

FIG. 6A illustrates one example of a method of determining which images from an intraoral scan are most likely to include caries, if caries are present in the teeth. This method may be performed prior to identifying caries within the scan.

FIG. 6B illustrates an example of a method of determining which images from an intraoral scan are most likely to include caries.

FIGS. 7A-7D illustrate one example steps that may be part of a method of determining images from an intraoral scan that are most likely to include caries.

FIG. 8 shows an example of one step that may be included as part of a method of determining images from an intraoral scan.

FIG. 9 schematically illustrates an example of features that may determine from images of an intraoral scan for scoring the images as described herein.

FIG. 10A shows an example of a 2D image having a relatively weak signal for a caries in the teeth.

FIG. 10B shows an example of a 2D image having two interproximal regions, each separately scored as described herein. One image, showing caries more clearly, has a higher score than the other region in the same 2D image.

FIGS. 11A and 11B illustrate an example of a method of forming a coverage map as described herein. FIG. 11A (left) shows a representation of the images before reducing the number of images (e.g., NIR and/or color images); FIG. 11B (right) shows the coverage map with the selected images indicated. FIG. 11B shows another example.

### DETAILED DESCRIPTION

In general the method and apparatuses described herein assist a user (e.g., dental professional, doctor, orthodontist, dentist, technical, etc.) in more efficiently reviewing and analyzing intraoral scan data. In particular, these methods and apparatuses, which may include devices and systems, including software, hardware and firmware, may be used with multiple modes of imaging, such as, but not limited to, visible light, including white light and narrow-band (red, green, blue, etc.) wavelengths, infrared/near-infrared light, fluorescent imaging, etc. Typical intraoral scans may include thousands, or even tens of thousands of images in a single scan lasting just a few minutes, and these scans may contain a vast amount of useful data that may be otherwise difficult, if not impossible, to manually review and interpret in an efficient manner. Thus the methods and apparatuses described herein address the technical problems arising because of the large amount of visual data generated by an intraoral scan, including identifying potentially rare events or indicators in the data, presenting the data in a compact and effective manner that bring appropriate attention to key features, and allowing the user to more effectively and efficiently review and interpret the data. Technical solutions to these technical problems are described in greater detail herein.

These methods and apparatuses may be configured to identify one or more images, or regions of images, from a plurality of images to that best show a feature or features of the patient's dentition, such as carries or other lesions. In particular, these methods and apparatuses may be used to identify optimal images or regions of images that best show one or more features even from images that include multiple instances of the feature, which may otherwise block or prevent the automated identification of optimal view of a feature in cases where multiple instances of the features are present.

For example, described herein are methods and apparatuses for determining which images may best show caries from an intraoral scan, should caries be present by scoring each scan image (e.g., the near-IR images and/or visible light images) to identify those that are most likely to best show the feature) e.g., caries. Alternatively or additionally, any of these methods and apparatuses may be configured to identify (following segmentation of a 3D model correlated to the plurality of images) regions likely to have the features, such as interproximal regions, and provide a score for each of the plurality of different regions on the images. Thus, the same image may have multiple scores corresponding to different regions within the image(s). The apparatus or method may use these region-specific scores to identify one or more images best illustrating the feature based on which region a user is interested in.

The methods and apparatuses described herein may be integrated into (or configured for use with) an intraoral scanner, and/or may be used separately from an intraoral scanner.

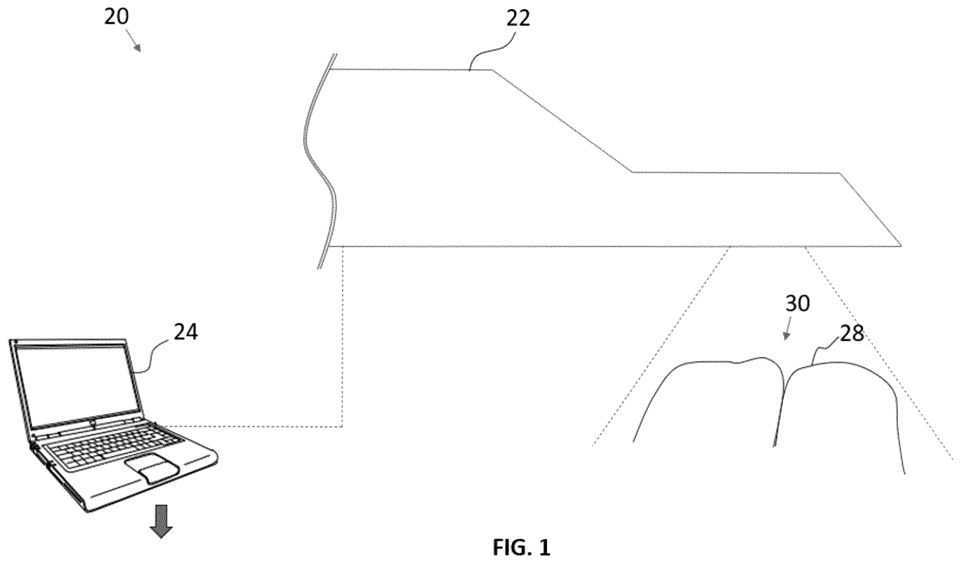

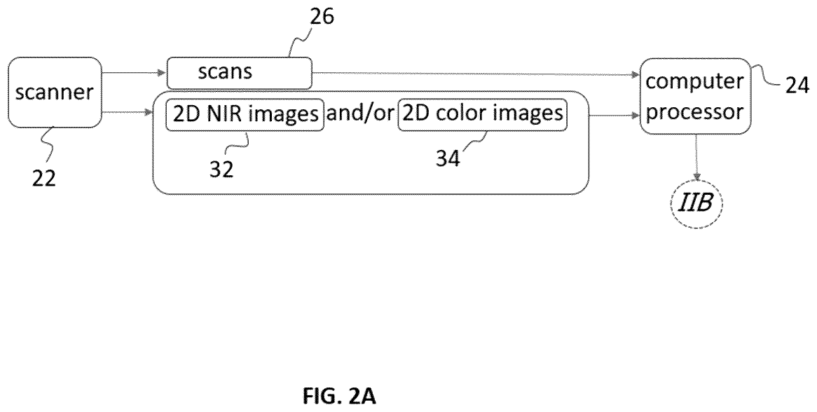

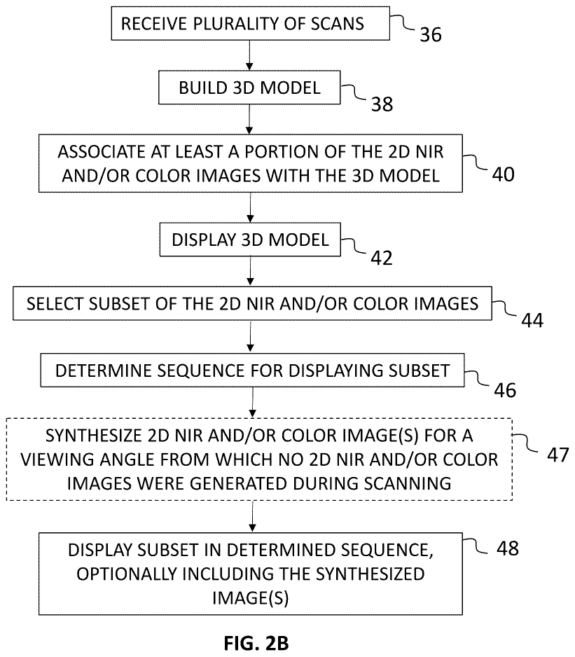

Reference is now made to FIGS. 1 and 2A-B. FIG. 1 is a schematic illustration showing an intraoral scanning system 20 including an intraoral scanner 22 and a computer processor 24, in accordance with some applications of the present invention. FIG. 2A shows a block diagram relating to the use of intraoral scanning system 20, in accordance with some applications of the present invention. Intraoral scanner 22 typically generates a plurality of scans 26 (FIG. 2A) of an intraoral three-dimensional (3D) surface 28 of an intraoral object 30 during intraoral scanning of intraoral 3D surface 28. For some applications, intraoral scanner 22 generates two-dimensional (2D) near-infrared (NIR) images 32 of intraoral object 30 during scanning of intraoral 3D surface 28.

Alternatively or additionally, for some applications, intraoral scanner 22 generates 2D color images 34 of intraoral 3D surface 28 during the intraoral scanning of intraoral 3D surface 28. For some applications, the 2D color images are images captured under white light illumination. Alternatively or additionally, the 2D color images are fluorescence images, e.g., images of intraoral 3D surface 28 fluorescing in response to ultraviolet (UV) illumination, or images of intraoral 3D surface 28 fluorescing in response to red light illumination.

FIG. 2B shows a flowchart illustrating steps performed by computer processor 24, in accordance with some applications of the present invention. For some applications, computer processor 24 is configured to: receive the plurality of scans 26 (step 36), build a 3D model of intraoral 3D surface 28 based on scans 28 (step 38) (or alternatively, receive a 3D model that has been previously assembled), associate at least a portion of 2D NIR images 32 and/or 2D color images 34 with the 3D model such that each of associated 2D NIR images 32 and/or associated 2D color images 34 corresponds to a position and viewing angle relative to the 3D model (step 40), display to a user the 3D model of intraoral 3D surface 28 (step 42), select a subset of the associated 2D NIR images 32 and/or associated 2D color images 34 that correspond to an area A of the 3D model (step 44), determine a sequence for displaying the selected subset of associated 2D NIR images 32 and/or associated 2D color images 34 (step 46), and display the selected subset of associated 2D NIR images 32 and/or associated 2D color images 34 in the determined sequence (step 48).

For some applications, step 48 includes displaying the selected subset on an area of the display that is separate from the 3D model, e.g., in a separate display window near where the 3D model is displayed. Alternatively, step 48 includes computer processor 24 displaying the selected subset by projecting the selected subset of associated 2D NIR images 32 and/or associated 2D color images 34 onto the 3D model.

Typically, during the scanning, many 2D NIR and/or 2D color images are generated as the scanner is moved around the intraoral cavity. Therefore, for each area A of the 3D model, there may be many different 2D NIR images 32 and/or 2D color images 34 to choose from, each corresponding to a position and viewing angle relative to the 3D model. For some applications, there may be more than one 2D NIR image 32 and/or more than one 2D color image 34 per viewing angle relative to the 3D model. Thus, for some applications, in step 44 computer processor 24 may consider the associated 2D NIR images 32 and/or associated 2D color images 34 corresponding to area A of the 3D model and select the best image from each viewing angle to be included in the subset to be displayed.

Note that within the 2D images, the same image may include multiple features relative to multiple different areas of interest (A). For example, the 2D images may be taken far enough from the teeth that multiple examples of the same class of feature (e.g., caries) may be visible (or likely to be visible) in the same image. In examples in which the feature being examined is the interproximal region and/or a carries likely to be located in or near an interproximal region of the teeth, a single 2D image may include multiple examples of interproximal regions of the teeth (which are statistically more likely to include carries). As described in greater detail below, these methods and apparatuses may identify multiple scores for different regions in the same image, and the select which score to use based on a proximity of the scored region to the area of interest, A.

As mentioned, in some cases the feature may be a dental pathology. For some applications, in step 44, computer processor 24 takes into account a dental pathology, e.g., caries, and selects images for the subset that will facilitate observation of the dental pathology, e.g., caries. For some applications, computer processor 24 uses image processing and/or machine learning techniques to perform an analysis of associated 2D NIR images 32 and/or associated 2D color images 34 corresponding to area A in order to determine which of the associated images would facilitate observation of the dental pathology.

Referring specifically to 2D color images 34, a factor that may affect the observation of a dental pathology, e.g., caries, in the images is the viewing angle relative to intraoral 3D surface 28 of each associated 2D color image. Thus, for some applications, computer processor 24 is configured to facilitate observation of a dental pathology, e.g., caries, by selecting the subset of associated 2D color images 34 based on respective viewing angles relative to intraoral 3D surface 28 of associated 2D color images 34.

For example, based on analysis of associated 2D color images 34 computer processor 24 may determine that the dental pathology is more easily viewed from a particular general viewing angle relative to intraoral 3D surface 28. Thus, in step 44 computer processor 24 may specifically select viewing angles that are very close to each other i.e., all close to the general viewing angle, and display to the user a few of the best images from that general viewing angle. Alternatively, for example, based on analysis of associated 2D color images 34, computer processor 24 may determine that the dentist should consider the tooth from multiple different viewing angles in order to observe the dental pathology. Thus, computer processor 24 may specifically select angles that are substantially different from each other, e.g., that pan around area A of the 3D model, e.g., going from the buccal side to the lingual side of area A, so that the dentist or dental technician sees a view of area A from different angles without having to rotate the 3D model on the display.

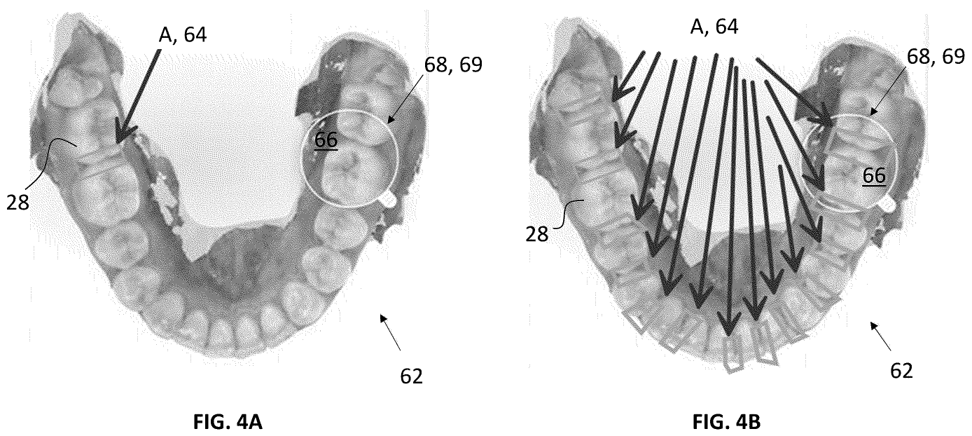

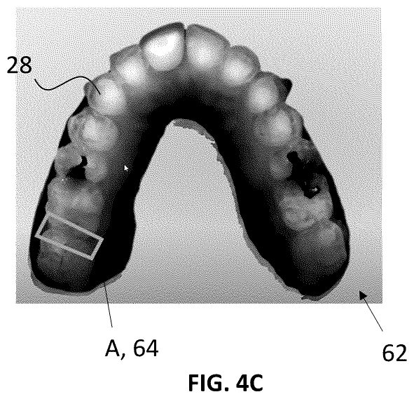

In any of these methods and apparatuses, the viewing angle may be derived from a user interface, such as the user interface including an area-selecting indicator, such as a viewing point or viewing window (e.g., loop, widow, etc.) as illustrated and described in FIGS. 4A-4B, below. In these cases the viewing angle may be derived from the area-selecting indicator.

Referring specifically to 2D NIR images 32, in addition to the viewing angle relative to intraoral 3D surface 28, the NIR-illumination angle is an additional factor which affects the ability to observe caries in a NIR image of intraoral object 30. The images of intraoral object 30 with occlusal NIR lighting may surprisingly have a higher chance of showing caries with good visibility. Thus, for some applications, in step 44 computer processor 24 facilitates observation of caries by selecting the subset of associated 2D NIR images 32 based on (a) respective viewing angles relative to intraoral 3D surface 28 of associated 2D NIR images 32 or (b) respective NIR-illumination angles relative to intraoral 3D surface 28 of associated 2D NIR images 32. For example, for a given area A of the 3D model, e.g., a tooth or interproximal area, computer processor 24 may consider an arc centered around area A spanning from the buccal side to the lingual side of area A. For each angle on the arc, all the 2D NIR images having a viewing angle that points toward the center of the arc are considered, and for each viewing angle the 2D NIR image having the most occlusal NIR-illumination angle is selected to be part of the subset that is displayed.

In general, the methods and apparatuses described herein may score all or some of the images of a scan, such as the near-IR image, visible light/surface images, etc., for one or more characteristic and may display and/or sort, and/or group images based on the score. Any appropriate characteristics may be scored, separately or together, such as, but not limited to, likelihood of showing caries, actually containing images caries, likelihood of showing cracking and/or wearing, actually showing cracking and/or wearing, clearest representation of a view of a tooth (e.g., from one or more perspectives, such as lingual, buccal, occlusal, etc.) in a particular wavelength (visible light, near-IR, fluorescent, etc.). These methods and apparatuses may use one or more characteristic from the image to score all or a sub-set of the images and may save the score, e.g., directly associated with the image or in an index referencing the image, for use, e.g., in displaying, sorting and/or analyzing the images. Characteristics that may be used to sort the images may be derived from the orientation of the one or more teeth in the image, the orientation of the gingiva, one or more optical properties such as luminosity, intensity, etc., and/or one or more imaging apparatus position and/or orientation (e.g., camera position, camera angle, camera distance, light source position, light source angle, distance between light source and tooth surface, distance between light source and camera, distance between camera and tooth surface, etc.). See, e.g., FIG. 9. In some cases one or more representative points or regions on the dentition (tooth, gingiva, etc.) may be used as a reference for one or more of these characteristics.

As mentioned above, all or some of the images may have multiple scores, associated with different regions of the same image. The score may be associated with a particular region or point on the image. The region or point may be identified from the associated 3D model. These regions may be identified by segmenting the 3D model, e.g., into teeth, gingiva, etc. Regions identified may include interproximal regions, teeth, etc. Thus, scores may be associated with these different regions and the images may be sorted (e.g., ordered, ranked, etc.), and/or selected based on the score specific to a region. In particular, based on proximity to the selected area from the user interface including proximity to the user-selectable area-selecting indicator (e.g., window, point, etc.), such as within the area, within a minimum distance (which may be user selectable or may be preset) of the area, or in some cases, the scored area that is closest to the user-selected area, A.

For example, described herein are methods and apparatuses for determining which images may best show caries from an intraoral scan, should caries be present, by scoring all or some of the images from a scan (e.g., the near-IR images and/or visible light images) and identifying those that are most likely to have caries, without necessarily identifying caries in the images. The images most likely to have carries may be presented to a user (e.g., displayed, saved, transmitted) and/or may be used as an input into any of these methods for identifying caries. Images may be selected based on the scores for the image or for regions of the images, and in particular, regions associated with a user-selected area, A.

For some applications, computer processor 24 automatically identifies a dental pathology or a suspected dental pathology, e.g., caries in intraoral object 30, based on the generated 2D color images 34. Similarly, for some applications, computer processor 24 automatically identifies caries or another suspected dental pathology in intraoral object 30 based on the generated 2D NIR images 32. For some applications, computer processor automatically selects area A of the 3D model based on the identification of the dental pathology, e.g., based on the identification of caries in intraoral object 30. For example, if computer processor 24 automatically identifies the presence of caries in a particular tooth within the intraoral cavity of a subject then computer processor 24 may automatically select that tooth, or more specifically the part of that tooth that has caries, as the area for which the subset of images is displayed to the dentist or dental technician. Alternatively or additionally, computer processor 24 may select the subset of associated 2D NIR images 32 and/or the subset of associated 2D color images 34 based on the identification of the dental pathology, e.g., based on the identification of caries in intraoral object 30.

For some applications, prior to displaying a selected subset of 2D NIR images 32 in step 48, computer processor 24 normalizes the selected subset of associated 2D NIR images 32 with respect to each other. Similarly, for some applications, prior to displaying a selected subset of associated 2D color images 34 in step 48, computer processor 24 normalizes the selected subset of associated 2D color images 34 with respect to each other. For example, computer processor 24 may normalize a selected subset of associated 2D NIR images 32 or associated 2D color images 34 by normalizing a brightness of at least one 2D NIR image 32 or 2D color image 34 of the subset with respect to a brightness of at least one other 2D NIR image 32 or 2D color image 34 of the subset. Alternatively or additionally, computer processor 24 may normalize a selected subset of associated 2D NIR images 32 or associated 2D color images 34 by normalizing a contrast within at least one 2D NIR image 32 or 2D color image 34 of the subset with respect to a contrast within at least one other 2D NIR image 32 or 2D color image 34 of the subset.

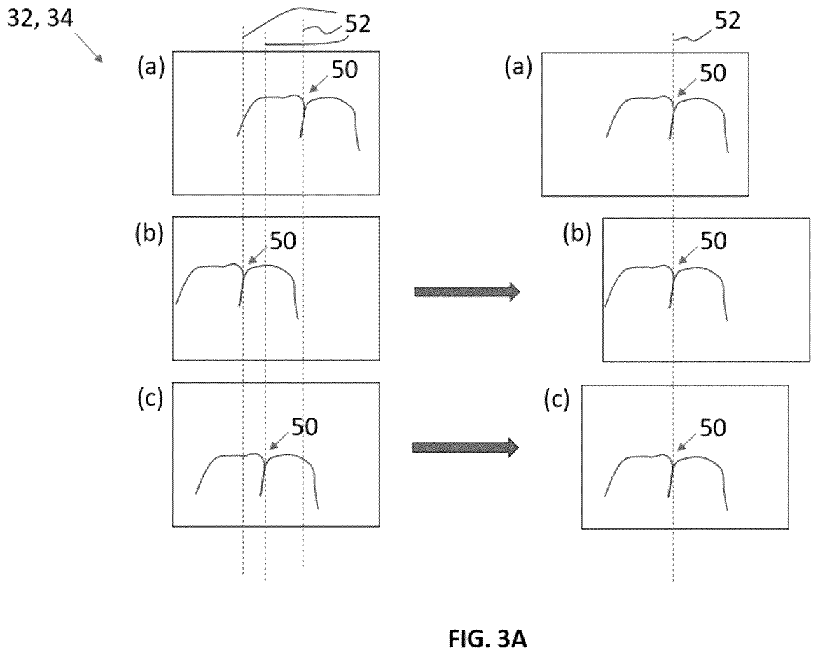

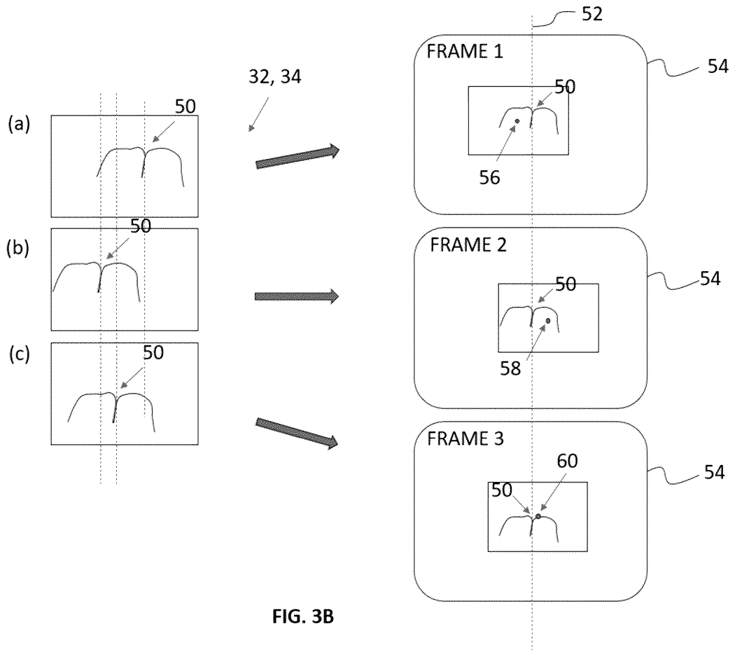

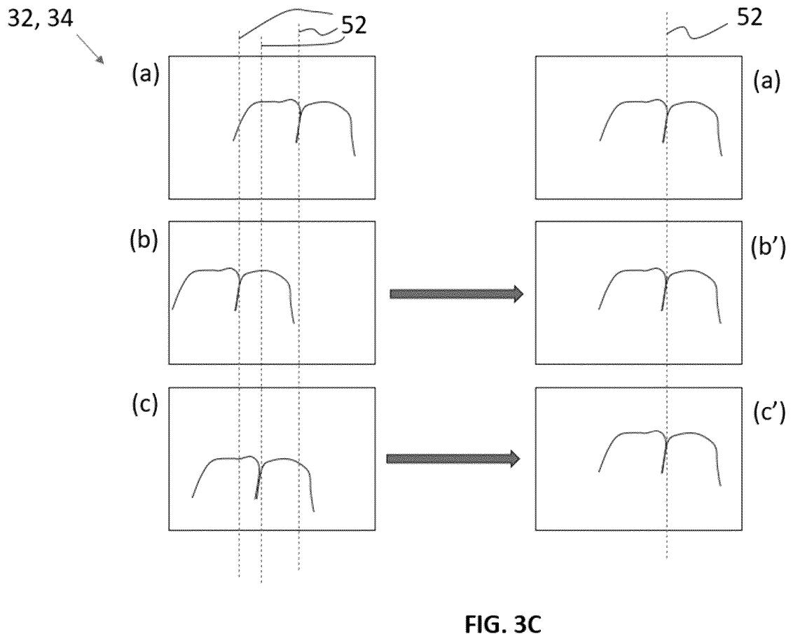

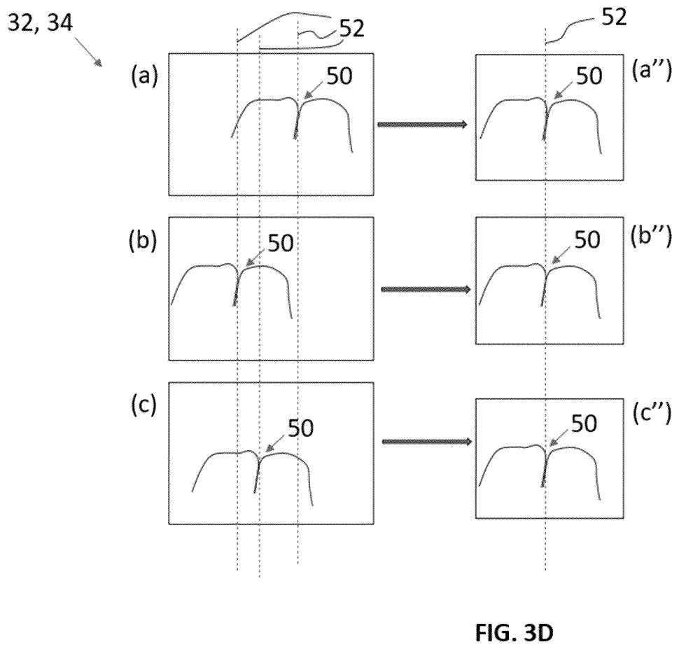

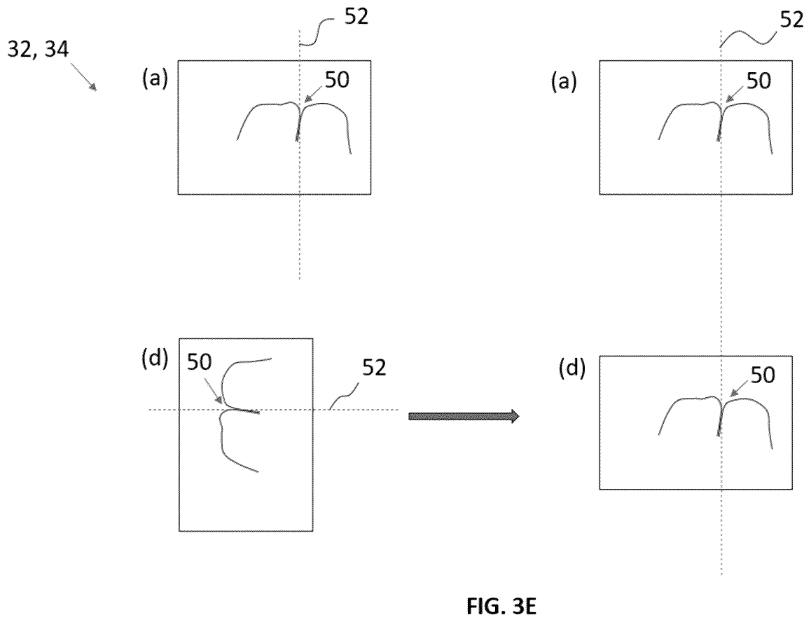

Reference is now made to FIGS. 3A-E, which depict various ways in which a common region of interest 50 as seen in each image of a selected subset of associated 2D NIR images 32 and/or associated 2D color images 34 is maintained spatially stable during transition by computer processor 24 from one image to the next within the selected subset, in accordance with some applications of the present invention. For some applications, the images selected by computer processor 24 to be included in the selected subset of associated 2D NIR images 32 and/or associated 2D color images 34 may not show the particular area of interest being considered in the same spatial location within each image of the subset. As a result, as computer processor 24 displays the selected subset in the determined sequence, the area of interest, e.g., an interproximal area between two teeth, may appear to jump around as computer processor 24 transitions from one image to the next within the selected subset.

Thus, for some applications, in step 48, computer processor 24 identifies a common region of interest 50 as seen in each image of the selected subset of associated 2D NIR images 32 and/or associated 2D color images 34. Computer processor 24 then displays the selected subset in the determined sequence while maintaining identified common region of interest 50 spatially stable during transitioning by computer processor 24 from one 2D NIR image to the next within the selected subset and/or from one 2D color image to the next within the selected subset.

As shown in FIG. 3A, for some applications, computer processor 24 maintains identified common region of interest 50 spatially stable during transitioning by aligning the selected subset of associated 2D NIR images 32 and/or associated 2D color images 34 with respect to each other. By way of illustration and example only, images (a), (b), and (c) shown on the left-hand side of FIG. 3A all show common region of interest 50 in different respective spatial positions within each image, as illustrated by dashed lines 52. The right-hand side of FIG. 3A shows images (a), (b), and (c) aligned so that as computer processor 24 transitions from image (a) to image (b) and then from image (b) to image (c), common region of interest 50 stays spatially stable, as illustrated by dashed lines 52 lining up with each other.

FIG. 3B shows an example of aligning common region of interest 50 when the images are displayed one at a time in the determined sequence on a display screen 54. This example uses the same three example images (a), (b), and (c) as used in FIG. 3A. The right-hand side of FIG. 3B shows three separate image frames on display screen 54, and in each image frame a separate one of the images (a), (b), and (c) is displayed. Thus, for some applications, computer processor 24 maintains identified common region of interest 50 spatially stable during transitioning by displaying at least a first image, e.g., image (a) in frame 1, of the selected subset centered around a first point 56 on display screen 54 and displaying at least a second image of the selected subset, e.g., image (b) in frame 2, centered around a second point 58, different from first point 56, on display screen 54. By way of example, FIG. 3B also shows image (c) in frame 3 centered around a third point 60, different from first point 56 and from second point 58 on display screen 54. Thus, even though the images themselves seem to jump around the display screen, when the images are displayed one after the other, the dentist or dental technician sees the region of interest maintained spatially stable.

FIG. 3C depicts another example of how computer processor 24 may maintain common region of interest 50 spatially stable during the transitioning. For some applications, computer processor 24 maintains identified common region of interest 50 spatially stable during transitioning by morphing at least one image of the selected subset of associated 2D NIR images 32 and/or associated 2D color images 34 to another one of the images. By way of example, FIG. 3C uses the same three example images (a), (b), and (c) as used in FIG. 3A. The left-hand side of FIG. 3C shows common region of interest 50 in different respective spatial positions within each image (a), (b), and (c), as illustrated by dashed lines 52. The right-hand side of FIG. 3C shows image (a) as being unchanged, however image (b) has been morphed by computer processor into image (b′) such that common region of interest 50 in image (b′) is in the same spatial location within image (b′) as it is within image (a). The example in FIG. 3C also shows image (c) morphed into image (c′), showing common region of interest 50 in image (c′) in the same spatial location within image (c′) as it is within image (a). It is noted that the morphing of the at least one image may be performed by computer processor 24 based on image processing and/or based on machine learning.

FIG. 3D depicts another example of how computer processor 24 may maintain common region of interest 50 spatially stable during the transitioning. For some applications, computer processor 24 maintains identified common region of interest 50 spatially stable during transitioning by cropping at least one image of the selected subset of associated 2D NIR images 32 and/or associated 2D color images 34. By way of example, FIG. 3D uses the same three example images (a), (b), and (c) as used in FIG. 3A. The left-hand side of FIG. 3D shows common region of interest 50 in different respective spatial positions within each image (a), (b), and (c), as illustrated by dashed lines 52. The right-hand side of FIG. 3D shows a cropped version of images (a″), (b″), and (c″), which have all been cropped such that common region of interest 50 is in the same spatial location within each of the cropped images.

FIG. 3E depicts another example of how computer processor 24 may maintain common region of interest 50 spatially stable during the transitioning. For some applications, computer processor 24 maintains identified common region of interest 50 spatially stable during transitioning by rotating at least one image of the selected subset of associated 2D NIR images 32 and/or associated 2D color images 34. The left-hand side of FIG. 3E shows common region of interest 50 in different respective spatial positions within each image (a) and image (d) as illustrated by dashed lines 52. The right-hand side of FIG. 3E shows image (d) having been rotated such that common region of interest 50 in rotated image (d) is in the same spatial location as in image (a).

It is noted that although each of the above examples depicts one way in which to maintain common region of interest 50 spatially stable during the transitioning from one image to the next within the selected subset, a combination of one or more of the above methods may be used.

Reference is now made to FIGS. 4A-C, which show a 3D model 62 of a dental arch of a subject after computer processor 24 has built 3D model 62 from scans generated during the intraoral scanning and has associated at least a portion of the generated 2D color images 34 (FIGS. 4A-B) and/or 2D NIR images 32 (FIG. 4C) with 3D model 62. For some applications, area A of 3D model 62 corresponds to an interproximal area 64 of intraoral 3D surface 28. For some applications, computer processor 24 automatically identifies one or more interproximal areas 64 of intraoral 3D surface 28, each identified interproximal area 64 corresponding respectively to an area A of 3D model 62. For example, if computer processor 24 has automatically identified a dental pathology, e.g., caries, in a particular interproximal area 64 (as described hereinabove), then the computer processor may automatically identify that interproximal area 64 for the dentist or dental technician to consider further. Another example is that since the interproximal areas of the dental arch are generally a common area for caries to form, computer processor 24 may automatically identify a plurality, e.g., all, of the interproximal areas on 3D model 62 (such as is shown in FIG. 4B) and select a subset of associated 2D NIR images 32 and/or associated 2D color images 34 to display for each of the identified interproximal areas.

For each automatically identified interproximal area 64, computer processor 24 may automatically select a subset of associated 2D NIR images 32 and/or associated 2D color images 34 that correspond to the respective area A of 3D model 62 (step 44, FIG. 2B). For each selected subset, computer processor 24 then performs steps 46 and 48, i.e., determining a sequence for displaying the selected subset of associated 2D NIR images 32 and/or associated 2D color images 34, and display the selected subset of associated 2D NIR images 32 and/or 2D color images 34 in the determined sequence.

Alternatively or additionally to automatic detection by computer processor 24 of one or more areas of interest on the 3D model, the dentist or dental technician can provide input regarding one or more given areas of the 3D model. Thus, for some applications, computer processor 24 receives user input, e.g., input by the dentist or the dental technician, associated with a given area 66 of 3D model 62 of intraoral 3D surface 28. In response to receiving the user input, computer processor 24 performs step 44 by selecting a subset of associated 2D NIR images 32 and/or associated 2D color images 34 that correspond to the given area of the 3D model. For example, given area 66 may be an interproximal area 64 of 3D model 62 corresponding to an interproximal area on intraoral 3D surface 28, and computer processor 24 receives the user input in response to the user selecting a particular one or more interproximal areas 64 on 3D model 62.

For some applications, computer processor 24 receives the user input by the user adjusting an indicator 68 on a view of 3D model 32. Given area 66 for which computer processor performs step 44 is an area of 3D model 32 that is associated with indicator 68. For some applications, indicator 68 is an area-selecting indicator 69, e.g., a loupe indicator such as is shown in FIGS. 4A-B. In this case, the user input is the user, e.g., the dentist or dental technician, moving area-selecting indicator 69, and the given area 66 is the area of 3D model 32 that is within area-selecting indicator 69.

Reference is again made to FIG. 2B. For some applications, it may be the case that for one or more given viewing angles there are no 2D NIR images 32 and/or no 2D color images 32 generated during the intraoral scanning. However, it may be helpful to the dentist or dental technician to see a smooth “video” displayed based on the selected subset of associated 2D NIR images 32 and/or associated 2D color images 34. Therefore, for some applications, in order to fill in the gaps in the “video” corresponding to “in-between” viewing angles for which no 2D NIR images 32 and/or no 2D color images 32 generated, computer processor 24 may synthesize one or more synthesized 2D color images and/or one or more synthesized 2D NIR images of intraoral 3D surface 28 based, respectively, on 2D color images 34 and/or 2D NIR images 32 generated during the intraoral scanning from nearby viewing angles (step 47). In step 48, computer processor 24 then displays the selected subset of associated 2D NIR images 22 and/or associated 2D color images 34 in the determined sequence by augmenting the selected subset with the one or more synthesized 2D NIR and/or the one or more synthesized color images respectively.

It is noted that techniques described in US20210068773 to Moshe et al., which is incorporated herein by reference, may be used for synthesizing the images. It is also noted that for some applications, a video clip made entirely of synthesized 2D NIR images and/or a video clip made entirely of synthesized 2D color images may be displayed. For example, if no good images were generated during the scanning for a particular area of the 3D model, then images may be synthesized for that particular area of the 3D model.

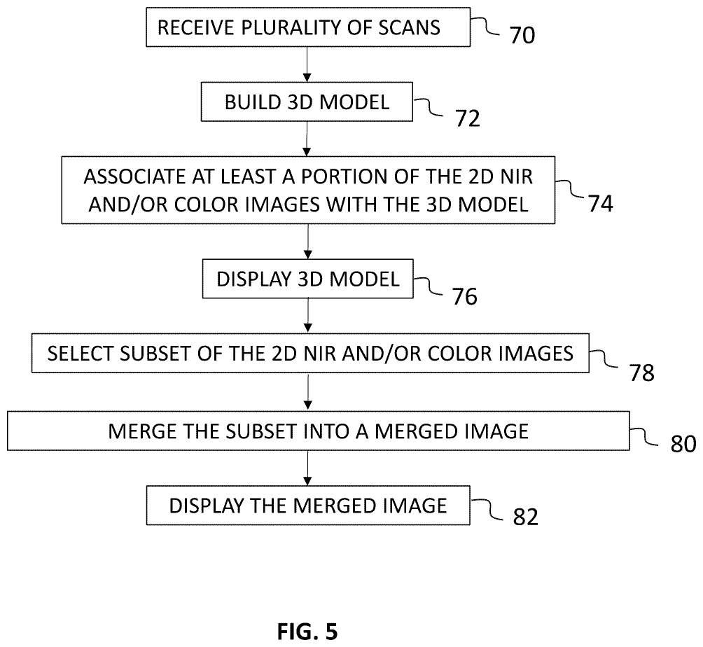

Reference is now made to FIG. 5, which is a flowchart illustrating steps performed by computer processor 24, in accordance with some applications of the present invention. For some applications, instead of displaying the selected subset of associated 2D NIR images 32 and/or associated 2D color images 34, computer processor 24 may merge the selected subset of associated 2D NIR images 32 and/or associated 2D color images 34 into a merged 2D NIR image and/or a merged 2D color image corresponding to area A of the 3D model (step 80 in FIG. 5). The merged image is then displayed to the dentist or dental technician (step 82 in FIG. 5). It is noted that steps 70, 72, 74, 76, and 78 in the method shown in of FIG. 5 are the same as steps 36, 38, 40, 42, and 44 of the method shown in FIG. 2B, mutatis mutandis. It is also noted that regarding step 78, when the selected subset of images are to be merged into a merged image, computer processor 24 typically selects a subset of associated 2D NIR images 32 and/or associated 2D color images 34 that are from the same general viewing angle.

Similarly to as described hereinabove, the merged 2D NIR image and/or the merged 2D color image may be displayed by being projected by computer processor 24 onto the 3D model.

It is noted that techniques described herein above relating to the following all apply to applications of the present invention in which the selected subset of images are merged into a merged image, mutatis mutandis:
(i) computer processor 24 facilitating the observation of a dental pathology, e.g., caries, based on the selection of the subset of associated 2D NIR images 32 and/or associated 2D color images 34,
(ii) computer processor 24 automatically identifying a dental pathology e.g., caries,
(iii) normalizing the selected subset of images, and
(iv) selection by computer processor 24 and/or based on user input of a given area of the 3D model, e.g., one or more interproximal areas.

For some applications, for a given area of the merged image, computer processor 24 may select specific sub-regions from each image of the selected subset to use for the given area of the merged image. For example, computer processor 24 can build the merged image by selecting “good” image portions from different images of the selected subset and assembling a merged image from the various “good” image portions.

Similarly to as described hereinabove with reference to FIGS. 3A-E, mutatis mutandis, for some applications, computer processor 24 (i) identifies common region of interest 50 as seen in each image of the subset of the associated 2D NIR images 32 and/or associated 2D color images 34, and (ii) aligns common region of interest 50 as seen in each image of the subset of associated 2D NIR images 32 and/or associated 2D color images 34 prior to merging the selected subset into the merged image. Furthermore, similarly to as described hereinabove, mutatis mutandis, computer processor 24 aligns identified common region of interest 50 as seen in each image of the selected subset by (i) cropping at least one image of the selected subset of the associated 2D NIR images 32 and/or associated 2D color images 34, (ii) rotating at least one image of the selected subset of the associated 2D NIR images 32 and/or associated 2D color images 34, and/or (iii) morphing at least one image of the selected subset of the associated 2D NIR images 32 and/or associated 2D color images 34 to the next.

For some applications, based on the aligning of common region of interest 50 as seen in each image of the selected subset of associated 2D NIR images 32 and/or associated 2D color images 34 prior to merging, computer processor merges the selected subset of the associated 2D NIR images 32 and/or associated 2D color images 34 by stacking the selected subset into a stacked image. Stacking the selected subset of images can increase image resolution within the merged image, thus creating a merged image in which it may be easier to identify a dental pathology, e.g., caries. Additionally, stacking the selected subset of images can increase signal to noise ratio within the merged image, which leads to better contrast within the merged image and which also may increase the ease with which a dental pathology, e.g., caries, can be identified. For some applications, for a given area of the stacked image, computer processor 24 may select specific sub-regions, e.g., specific groups of pixels, from each image of the selected subset to use for the given area of the stacked image.

For some applications, intraoral scanner 22 generates 2D NIR images 32 and 2D color images 34 during the intraoral scanning of intraoral 3D surface 28. In step 74 computer processor 24 (i) associates at least a portion of 2D NIR images 32 with the 3D model such that each of associated 2D NIR images 32 corresponds to a position and viewing angle relative to the 3D model, and (ii) associates at least a portion of 2D color images 34 with the 3D model such that each of associated 2D color images 34 corresponds to a position and viewing angle relative to the 3D model. In step 78 computer processor 24 then (i) selects a subset of associated 2D NIR images 32 that correspond to an area A of the 3D model, and (ii) selects a subset of associated 2D color images 34 that correspond to the same area A of the 3D model. In step 80 computer processor 24 merges the selected subset of associated 2D NIR images 32 with the selected subset of associated 2D color images 32 to create a merged 2D NIR-color image corresponding to the area A of the 3D model. The merged 2D NIR-color image is then displayed to the dentist or dental technician in step 82.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

As mentioned, also described herein are methods and apparatuses (e.g., intraoral scanners) that are configured to identify which images of an intraoral scan, including which near-IR and/or visible light images, have one or more properties and score them, then use the score to display, sort and/or analyze the images. For example, these methods and apparatuses may be used to rank images of a scan, typically after the scan has been completed, though in some cases the method may be performed in an ongoing manner, as scans are being collected. In one example, described in detail below, the images may be scored based on the likelihood that the images show caries. Thus, the ranking may indicate the likelihood of an image (e.g., a 2D image from the scan) showing a caries even without having to actually identify caries in the image(s). The resulting rankings may be used to display images for manual review, e.g., by a user, and/or for automatic detection of caries, as described above.

Although the example described below is specific to ranking based on the likelihood that the images may show a caries, one or more other characteristic may be used to score the images. For example, images may be scored based on how clearly the image shows a feature of the tooth or gingiva (e.g., crack, wear, grinding, abscess, etc.) and/or how representative the image shows the teeth or gingiva (e.g., a buccal, lingual or occlusal view, etc.), or the like. These methods may dramatically simplify, enhance and speed-up the review and analysis of the intraoral scan of a subject's dentition.

Near-IR images, in particular, may be difficult to interpret given the complexity of the way in which light traverses the interior of the tooth, resulting in different signal behaviors for near-IR images. As a result, caries may be visible in some images but not readily visible in other images of the same tooth surface. Surprisingly, the ability to visualize caries in near-IR images may be predicted based on properties of the tooth (tooth surface) and imaging system (e.g., camera and illumination source) and the relationship between the two. The methods and apparatuses described herein may therefore score or rank images from a scan (e.g., an intraoral scan) based on these properties in order to identify a subset of images form the intraoral scan that are most likely to show visible caries (if the tooth being imaged actually contains a caries). Thus, some near-IR images are more likely to allow caries to be seen in the image than others. In one example, the near-IR images from a plurality of near-IR images may be processed as described herein in order to determine a likelihood that a caries would be visible from that particular near-IR image; this prediction may be based on one or more features describing a relationship between one or more reference points in an interproximal region of a tooth in the near-IR image and one or more camera parameters of a camera that is used to take the near-IR image. This may be achieved by identifying, for each image of the plurality of near-IR images, one or more reference points in an interproximal region of a tooth, and for each one or more reference point, identifying a normal to the reference point relative to the tooth, and a camera angle of the camera taking the near-IR images, and estimating an angle between the normal and the camera angle. In other examples, the accuracy of the prediction may be increased by increasing the parameters or properties used to score the image, beyond just the angle between the normal and the camera angle, as will be described in greater detail herein. In this first example, the property that is used to determine a score of how likely a carious lesion (e.g., caries) will be visible in the near-IR image may be based on the camera angle relative to a surface normal of the tooth.

Because there can be hundreds, or even thousands of near-infrared (near-IR) images taken as part of a single scan, it would be extremely helpful to select a subset of images that are most likely to show caries based on a likelihood score, as described herein. The scored images may be used to determine which images should be displayed to the user (e.g., doctor, dentist, dental professional, technician, etc.) and/or which subset of images should be used in any of the caries identification algorithms described herein. Thus, images having a sufficiently high score may be shown to the user only after this scoring/selection process. The score may be relative to the other images, or they may be independent of the other images.

Different scoring techniques may be used. For example, a first scoring technique may be based on tooth orientation in relation to the near-IR light source and/or camera position, as mentioned above. Alternatively or additionally, the scoring may be instead based on a trained machine learning agent that uses multiple inputs or properties which may be taken or measured from the image(s). For example, a machine learning agent trained as a classifier may be used by training on properties such as geometrical features of the tooth/tooth surfaces and grey level information from the near-IR images.

In general, there methods and apparatuses may be configured to grade the near-IR images to determine a likelihood that these images will show visible caries in the near-IR image. In cases where the images having a high likelihood of showing a caries do not include a caries, this may provide a useful negative control for the user. In general, grading the near-IR images with a likelihood for each image that it will show caries (or will develop into caries) may be more efficient, e.g., more quickly performed, and may be performed as part of the apparatus requiring less time and computational power than analyzing these images to identify actual or potential caries. Further, even in cases in which caries may be present, it may be difficult to see the caries in some images, given the lighting/position of the caries. Thus, these methods may be performed in combination with any of the caries detection techniques discussed above, in order to show to the user the best image (e.g., the images most likely to include a visible caries. In some cases the score/grading mechanism may be used as part of an entirely local process that may be part of the intraoral scanner itself and may not require a remote processor.

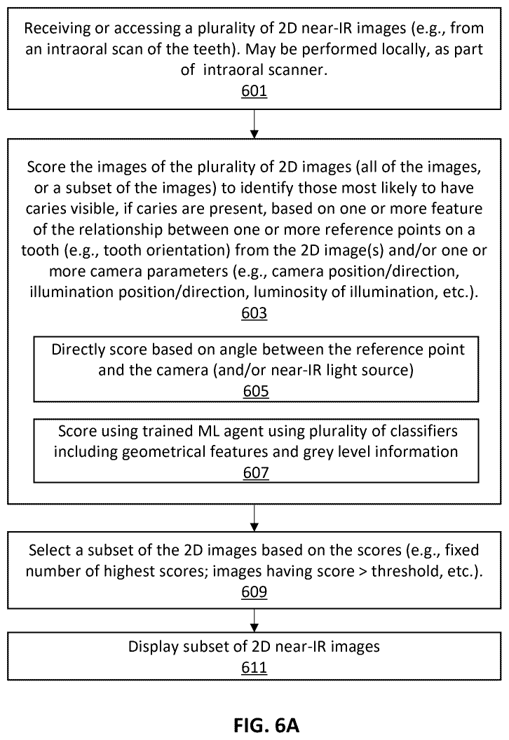

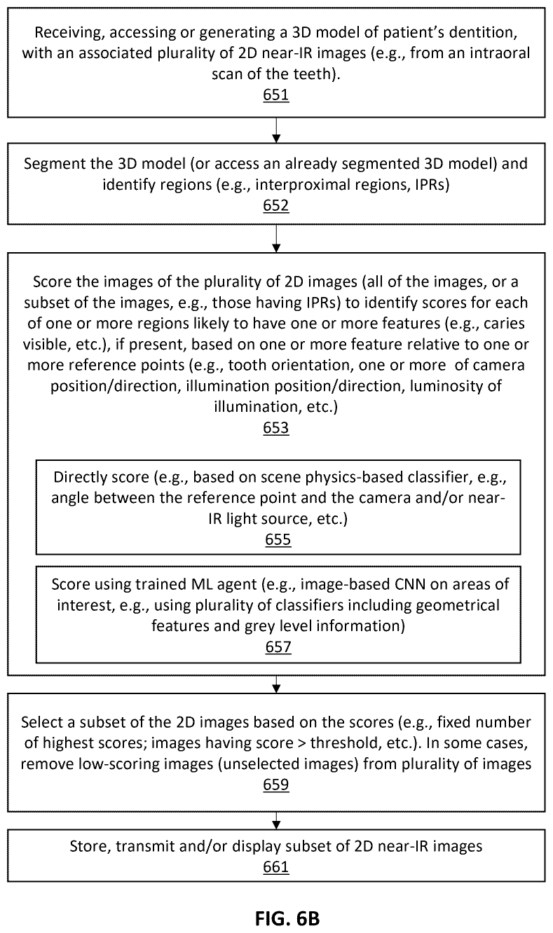

For example, FIG. 6A illustrates a schematic example of a method of identifying near-IR images that are most likely to show a caries. As shown in FIG. 6A, The method may include receiving (and/or accessing) a plurality of 2D near-IR images, e.g., from an intraoral scan of the subject's dentition. 601 The processor(s) performing this method may be part of the intraoral scanner (e.g., may be local). As mentioned, because this technique may be performed quickly and requires low computational power, particularly as compared with methods requiring identification of actual caries, it may be readily performed as part of the intraoral scanner.

The images may then be scored 603. All or a subset of the images of the plurality of 2D images may be scored to identify those most likely to have caries visible, if caries are present. Scoring may be relative to an absolute scale (e.g., regardless of the scores of the other images) or may be relative to the other images from the scan. In some cases the scores may be normalized to the other images. In general, scoring may be based on one or more feature of the relationship between one or more reference points on a tooth (e.g., tooth orientation) from the 2D image(s) and/or one or more camera parameters (e.g., camera position/direction, illumination position/direction, luminosity of illumination, etc.). The images to be scored may be the original images, or they may be modified from the original scan (e.g., filtered, smoothed, interpolated, etc.). The images may be near-IR images. In some cases the images may be or may include color images, fluorescent images, or other imaging types, or combinations of these (e.g., an image showing both visible light and near-IR, for example).

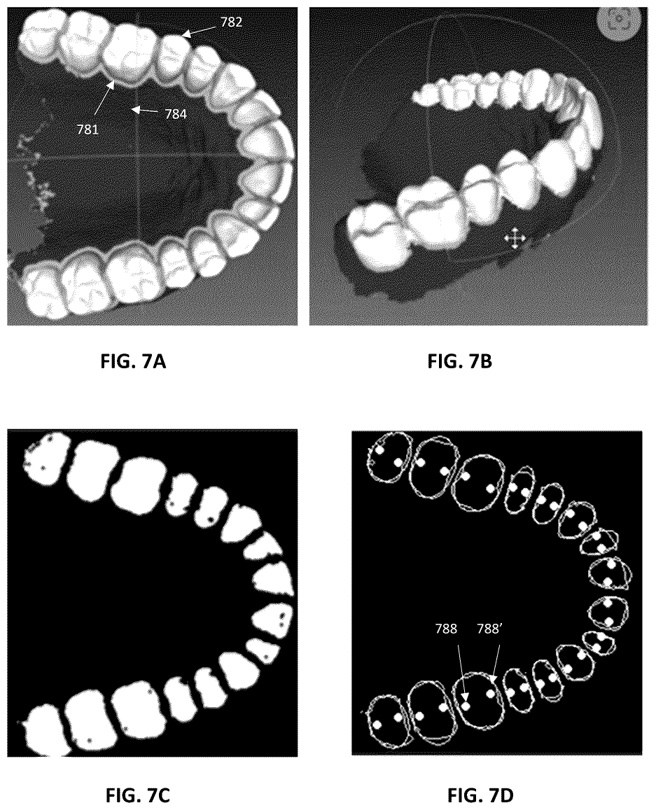

Prior to scoring, the images may be prepared. For example as each image is scored, it may be analyzed, filtered, etc. In some cases the images may be first examined to identify the one or more reference points, e.g., in the interproximal region. For example, one or more reference points may be identified on each tooth, such as a reference point from the distal (e.g., distal interproximal) interproximal region and/or a reference point from the medial (e.g., medial interproximal) region of each tooth. The reference points may be identified using the 3D model of the teeth (e.g. digital model, such as a digital surface model of the subject's dentition). The one or more reference points may be mapped (in some cases from the 3D digital model) to the 2D image(s), such as the near-IR images. FIGS. 7A-7D, discussed in detail below, show one example of a method of identifying one or more reference points.

In some cases scoring may be performed by determining, for each of the one or more reference points present in the 2D image, using one particular feature that is well-correlated with the presence and/or absence of dental caries. For example, scoring may include directly scoring the images based on angle between the reference point and the camera (and/or near-IR light source) 605. In this case, the feature is the relationship, e.g., the angle, between a normal on the tooth surface at the reference point, providing tooth orientation, and the camera angle (e.g., a camera parameters that includes the direction of the camera).

Alternatively in some cases multiple different parameters may be used, including some that relate more specifically to the relationship between the tooth (tooth orientation) and the imaging system (e.g., camera, lighting, etc.) or relate to the imaging system (e.g., relationship between the camera and the light source(s)), and/or the tooth. These multiple parameters may be used as classifiers for use with a trained machine learning agent (e.g., algorithm) 607. For example a trained ML agent may use a plurality of classifiers including geometrical features and grey level information to determine a score. Any number of different classifiers may be used. As a non-limiting example, the classifiers that may be used may include one or more of: cosine of the angle between camera and normal at reference point, Z absolute value of reference point to camera vector; camera to reference point distance in mm; cosine of the angle between camera direction and reference point; absolute of z value of camera direction vector; cosine of the angle between camera refraction and tooth axis; absolute z value of light source (e.g., LED) direction vector; cosine of the angle between a first light source (e.g., LED1) and normal; cosine of the angle between LED1 direction and reference point; distance between LED1 location and reference point in mm; distance between LED1 location and camera in mm; LED1 luminosity; absolute of z value of reference point to LED1 vector; cosine of the angle between a second light source (e.g., LED2) direction and normal; LED2 luminosity; absolute of z value of reference point to LED2 vector; cosine of the angle between LED2 direction and reference point; distance between LED2 location and reference point in mm; and/or distance between LED2 location and camera in mm.

Once scored, the scores may be adjusted (e.g., normalized) and may be used to filter or otherwise sort the images and may be used to generate one or more sub-sets of images. For example, these methods may select a subset of the 2D images based on the scores. The higher scores may correspond to a greater likelihood of seeing a dental caries on the image. In general, the higher scoring images may be displayed to the user (e.g., doctor, clinician, dentist, orthodontist, etc.). The higher scoring images may be selected for display and/or saving (e.g., as a subset of the total images in the scan) 609. In some cases those images having a score that is greater than a minimum threshold may be displayed. If scores as between 0 and 1.0 or any other range, the threshold may be adjusted so that only the highest x percent (e.g., 90%, 95%, 96%, 97%, 98%, 99%, 99.1%, etc.) are shown or selected for display. In some case the threshold may be set and/or adjusted by the user (e.g., in a user display/interface, the user may select, or adjust, this threshold up/down). In some cases the method or apparatus may simply display the top y number of most likely e.g., a fixed number of highest scores; images having score>threshold, etc.). Alternatively or additionally, the higher-scoring images may be displayed 611. Thus, in any of these examples a separate selection step, 609, is not required, but instead the scored images may be directly displayed, e.g., in a user interface.

The scores may be stored (e.g., in a data field, as metadata, etc.) and/or an index of the scores including the corresponding images or reference to uniquely identify the images, may be stored locally and securely by or within the intraoral scanner and/or a remote server. As mentioned here, any appropriate user interface and display may be used, including showing the high-scoring images individually or as a group or groups. The user may switch between different images, manually or automatically. In some cases, the user may select region or teeth (e.g., on a 3D model/display, including a 3D surface model) and be shown a 2D image having a high score that corresponds to the selected tooth or teeth (ore region). The 2D image may include markings indicating the presence of a high-scoring 2D image that is or can be, displayed. In some cases, images that do not have a sufficiently high score (e.g., below a threshold) may not be shown, and/or may be “discarded” from the user interface. Alternatively, in some cases the user may select to specifically be shown some or all of the lower-scoring images.

As mentioned, in some cases the method or apparatus may use the angle between a representative point and the camera and/or light source as a parameter, either on its own or as a parameter input into a trained machine learning agent. For example, the method or apparatus may be configured to compute a score that is based on the angle between a normal to the surface of the tooth at the selected representative tooth and the primary transmission angle of a light source emitting the light used to capture the image (e.g., one or more LED that is used for transmitting the near-IR light to the teeth). In some cases multiple light sources may be used. The location of the light from the light source and the position of light in relation to the possible carious lesion may impact the visibility of the caries. Thus the score may be implemented based on an angle that is formed between the normal and the source of light from the LED; for example, the smaller the angle, the higher the score. In cases where very close or near images are scored similarly high, near-duplicate or very similar and/or overlapping images may be removed or marked as duplicate (and may not need to be displayed). In some cases only on high-scoring image (e.g., highest scoring image) may be selected and/or displayed.

As mentioned, any of these methods and apparatuses may include determining a representative or reference point, also referred to as an anchor point, that may be used as a basis of calculation. Since the location of an actual carious lesion is not known, these reference points may be broadly selected as areas where the carious lesion may be, for example, within the interproximal (IP) region of a tooth. This reference point may be used to determine a normal (surface normal) from the tooth.

Any of these methods and apparatuses for performing them described herein may be configured to handle images having multiple candidate regions for the feature (e.g., caries) being detected and/or scored, e.g., automatically (and preferably rapidly, e.g., in near real time or real time). These methods may, in some cases, select optimal 2D images from a plurality of images by using both a scene classifier (e.g., a physics-based scene classifier) and a machine learning agent (e.g., a convolutional neural network, CNN) to determine which images best show the optimal images. Thus, by using scene physics (e.g., as part of a scene classifier), as well as, and in some cases, in parallel with, image classification techniques such as focusing on local regions using a CNN, accurate and fast optimal image selection may be achieved.

For example, FIG. 6B illustrates a schematic example of a method of identifying near-IR images that are most likely to show a caries, similar to that shown in FIG. 6A, but including details showing how multiple regions of the same 2D images may be used. In FIG. 6B the method may include receiving and/or accessing and/or generating 3D digital model of a patient's dentition 651. The 3D model includes a plurality of associated 2D image (e.g., near-IR images), which may be taken from an intraoral scan of the subject's dentition. The 3D model may already be segmented or may be segmented upon receipt/generation. For example, the 3D digital model may be segmented to identify teeth, gingiva, etc. and/or may identify interproximal regions (IPRs). In some cases the processor(s) performing this method may be part of the intraoral scanner (e.g., may be local or remote, as mentioned in reference to FIG. 6A).

The images may then be scored 603, including scoring multiple regions corresponding to regions identified from the 3D model, such as (but not limited to) interproximal regions.

As mentioned, prior to scoring the images may be prepared. For example as each image may be scored in one or more (e.g., two, three, four etc.) regions. The scored images may be analyzed, filtered, etc. In some cases the images may be first examined to identify the one or more reference points, e.g., in the interproximal region(s). For example, one or more reference points may be identified in the image(s) on each tooth within the 2D image, such as a reference point from the distal (e.g., distal interproximal) interproximal region and/or a reference point from the medial (e.g., medial interproximal) region of each tooth. The reference points may be identified using the 3D model of the teeth (e.g. digital model, such as a digital surface model of the subject's dentition). The one or more reference points may be mapped (in some cases from the 3D digital model) to the 2D image(s), such as the near-IR images. Once identified, each region may be scored.

All or a subset of the images of the plurality of 2D images may be scored to identify those most likely to have caries visible, if caries are present. Multiple different regions of each 2D image may be independently scored. As described in reference to FIG. 6A, scoring may be relative to an absolute scale (e.g., regardless of the scores of the other images) or may be relative to the other images from the scan. In some cases the scores may be normalized to the other images. In general, scoring may be based on one or more feature of the relationship between one or more reference points on a tooth (e.g., tooth orientation) from the 2D image(s) and/or one or more camera parameters (e.g., camera position/direction, illumination position/direction, luminosity of illumination, etc.). The images to be scored may be the original images, or they may be modified from the original scan (e.g., filtered, smoothed, interpolated, etc.). The images may be near-IR images. In some cases the images may be or may include color images, fluorescent images, or other imaging types, or combinations of these (e.g., an image showing both visible light and near-IR, for example). The physical scene of the image may be determined as described above. For example, the modeling of the physical scene may include the camera view, LED (light source) position(s), surface properties (e., normal, curvature, etc.) 655. This physical description may be extracted and may be used as described herein. In any of these methods, the use of machine learning, such as a combinatorial neural network, may be used in sequence with, or more preferably in parallel with the physics-based classifier 657.

Scoring may be performed by determining, for each of the one or more reference points present in the 2D image, using one particular feature that is well-correlated with the presence and/or absence of dental caries. For example, scoring may include scoring based on scene physics (“directly scoring” the images), e.g., based on angle between the reference point and the camera (and/or near-IR light source) 655. In this case, the feature is the relationship, e.g., the angle, between a normal on the tooth surface at the reference point, providing tooth orientation, and the camera angle (e.g., a camera parameters that includes the direction of the camera). Alternatively or additionally, multiple different parameters may be used, including some that relate more specifically to the relationship between the tooth (tooth orientation) and the imaging system (e.g., camera, lighting, etc.) or relate to the imaging system (e.g., relationship between the camera and the light source(s)), and/or the tooth. In some examples, these multiple parameters may be used (e.g., instead of or, preferably, in addition to) as classifiers for use with a machine learning agent (e.g., algorithm), such as a CNN 657. For example a trained ML agent may use a plurality of classifiers including geometrical features and grey level information to determine a score. Any number of different classifiers may be used. As a non-limiting example, the classifiers that may be used may include one or more of: cosine of the angle between camera and normal at reference point, Z absolute value of reference point to camera vector; camera to reference point distance in mm; cosine of the angle between camera direction and reference point; absolute of z value of camera direction vector; cosine of the angle between camera refraction and tooth axis; absolute z value of light source (e.g., LED) direction vector; cosine of the angle between a first light source (e.g., LED1) and normal; cosine of the angle between LED1 direction and reference point; distance between LED1 location and reference point in mm; distance between LED1 location and camera in mm; LED1 luminosity; absolute of z value of reference point to LED1 vector; cosine of the angle between a second light source (e.g., LED2) direction and normal; LED2 luminosity; absolute of z value of reference point to LED2 vector; cosine of the angle between LED2 direction and reference point; distance between LED2 location and reference point in mm; and/or distance between LED2 location and camera in mm.

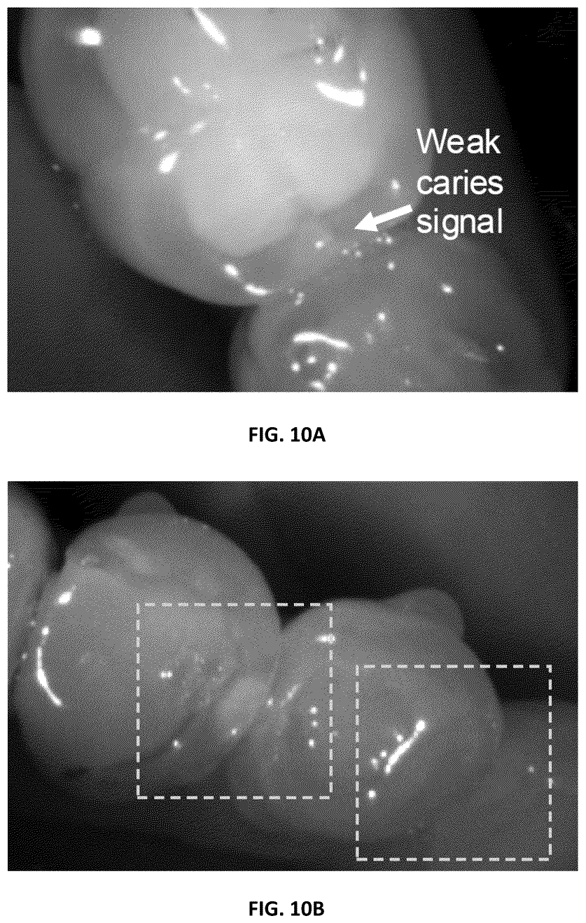

As mentioned, scoring may be performed for different regions on the same image (e.g., in some examples, where the image includes multiple interproximal regions). For example, FIG. 10A shows an example of an image having an interproximal region (single interproximal region) and, as indicated by the arrow, a weak signal for caries. FIG. 10B shows an example of an image having two interproximal regions (boxed), one of which shows a stronger signal for caries than the other.

Once scored, the scores may be adjusted (e.g., normalized) as described above. Higher scores may correspond to a greater likelihood of seeing a dental caries on the scored region of the image. In general, the higher scoring images may be displayed to the user (e.g., doctor, clinician, dentist, orthodontist, etc.). The higher scoring images may be selected for display and/or saving (e.g., as a subset of the total images in the scan) 659. In some cases those images having a score that is greater than a minimum threshold for the scored that is within a predetermined distance from a user-selected area, A, as described above. If scores for such regions as between 0 and 1.0 or any other range, the threshold may be adjusted so that only the highest x percent (e.g., 90%, 95%, 96%, 97%, 98%, 99%, 99.1%, etc.) are shown or selected for display. Regions outside of the user-selected area, A, are not considered/included. In some case the threshold may be set and/or adjusted by the user (e.g., in a user display/interface, the user may select, or adjust, this threshold up/down). In some cases the method or apparatus may simply display the top y number of most likely e.g., a fixed number of highest scores; images having score>threshold, etc.). Alternatively or additionally, the higher-scoring images may be displayed 661. Thus, in any of these examples a separate selection step, 659, is not required, but instead the scored images may be directly displayed, e.g., in a user interface.

The scores may be stored (e.g., in a data field, as metadata, etc.) and/or an index of the scores including the corresponding images and/or regions of the image or reference to uniquely identify the images, may be stored locally and securely by or within the intraoral scanner and/or a remote server. As mentioned here, any appropriate user interface and display may be used, including showing the high-scoring images individually or as a group or groups. The user may switch between different images, manually or automatically. In some cases, the user may select region or teeth (e.g., on a 3D model/display, including a 3D surface model) and be shown a 2D image having a high score that corresponds to the selected tooth or teeth (ore region). The 2D image may include markings indicating the presence of a high-scoring 2D image that is or can be, displayed. In some cases, images that do not have a sufficiently high score (e.g., below a threshold) may not be shown, and/or may be “discarded” from the user interface. Alternatively, in some cases the user may select to specifically be shown some or all of the lower-scoring images.

FIGS. 7A-7D illustrate one example of a technique for identifying the reference point(s) on each tooth from the scan. As mentioned a medial and proximal point may be determined. The method and/or apparatus may estimate (e.g., calculate, approximate, etc.) the interproximal region from a segmented image or model (e.g., 3D model) of the teeth. FIGS. 7A and 7B show a 3D digital model of the dentition that has been segmented into teeth 782 and gingiva 784. The interproximal region 781 is highlighted. Segmentation may identify these different regions and parts. If a 3D model is used to identify these points the corresponding regions on the 2D images (near-IR images) may be identified and used.

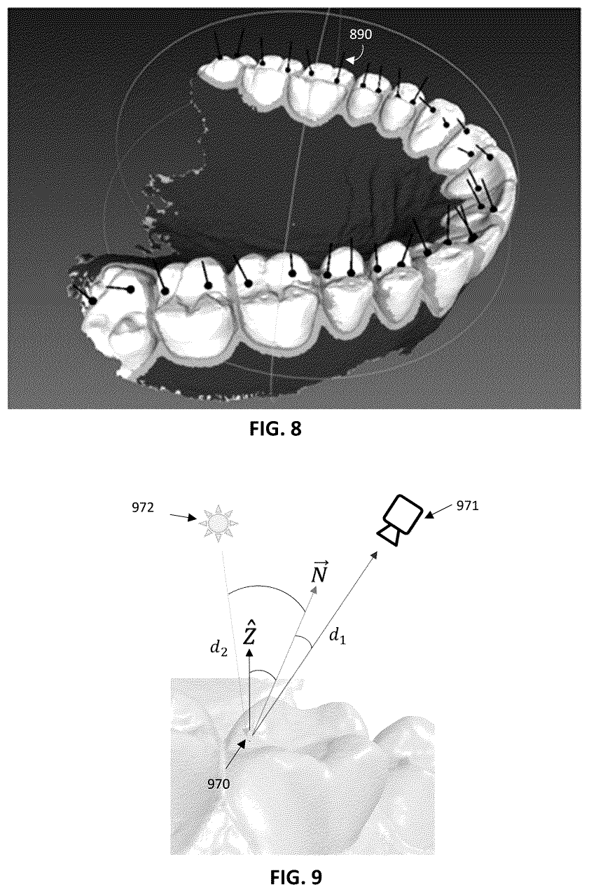

The segmented surface images/models shown in FIGS. 7A-7B may be used to identify interproximal regions, as shown in FIG. 7C, and from these images/models reference points 788, 788′ (two for each tooth) may be determined, as shown in FIG. 7D, and used so that for each image, e.g., each near-IR image, in the scan, the reference points may be used in to determine the orientation of the tooth or tooth surface, which can be used to determine one or more property. For example, in FIG. 8, an average normal 890 to the tooth surface may be determined around each point. This normal may be used to determine the single classifier (e.g., the angle between the camera angle and/or light source) and/or the some of the multiple classifiers that may be used to determine the score as described above. In any of these methods and apparatuses, the classifiers may be based on physics (e.g., scene physics). For example, in one non-limiting example, the score may be based on a trained ML agent that uses 19 geometrical features as classifiers that describe the relationships between the camera, LEDs, and interproximal reference points. These classifiers may include refraction, the relative angles between the camera angle, light source(s), and the tooth surface, LED luminosity, etc. For example, refraction (e.g., refraction of a camera ray to IP reference point) may be used. In some cases LED luminosity, e.g., calculated as fraction of light emitted in the IP point direction compared to portion of light that is projected directly, may be used. FIG. 6 illustrate some of the relationships between the teeth (e.g., representative point 670), camera 671, a light source 672.

In practice these methods and apparatuses for predicting images that may best show a carious lesion (caries) may be used before actual caries are detected. This may provide an overall screen that may indicate (e.g., based on the number of images above threshold) the likelihood that caries are present. This may also be used to assist in manually or automatically detecting caries, e.g., using image above a predetermined threshold for the score. For example, these methods may be performed post-processing. The actual scores (e.g., numeric scores) may or may not be displayed and may dramatically simplify and shorten the review process for images, and in particular near-IR images. In some examples a typical scan including near-IR images may record over a thousand images (e.g., between 3-6 thousand images). These techniques may determine the small subset of such images that are likely to show caries from these near-IR images, which may be much less than 1% of the total images collected.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

### Example

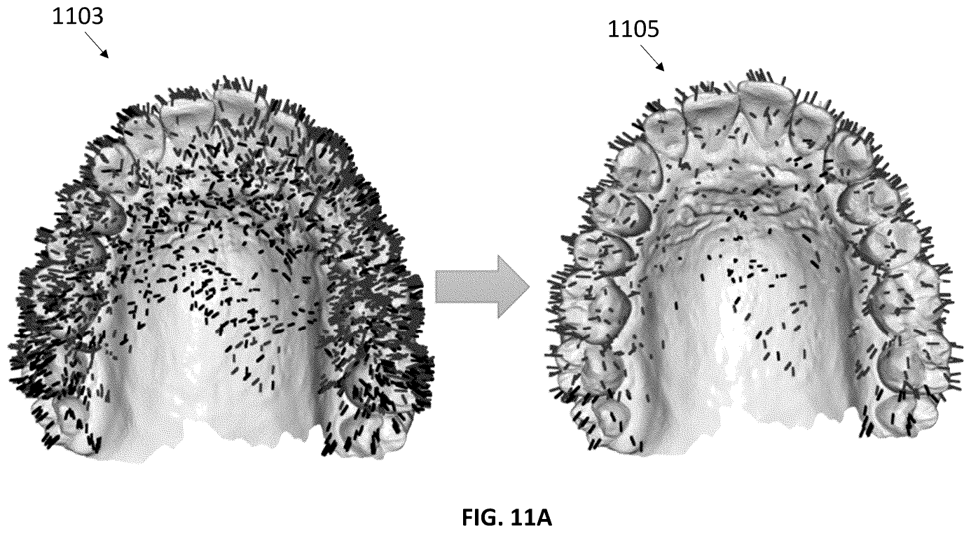

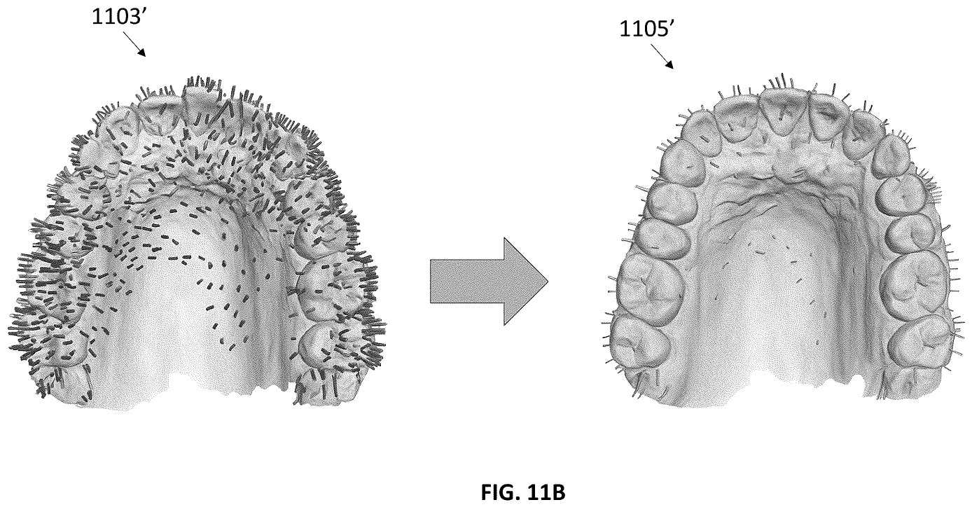

FIGS. 11A-11B graphically illustrate the formation of a coverage map using the techniques described herein. As described above, the methods and apparatuses described herein may be particularly helpful in reducing the sizes of large collections of files, and in particular collections of images by scoring the individual images (or regions/subregions within the images) to indicate a likelihood that these images (or one or more regions within the image(s)) include information about one or more features (e.g., caries, interproximal regions, lesions, etc.) and using these scores to either (or both) select a subset of image that are most likely, above some threshold, to include information about one or more features and/or remove images from a collection (e.g., a plurality, a set, a directory, a file, etc.) of images to form a reduced set or sub-set of image. This reduced set of images may be stored, transmitted and/or displayed; in any of these cases the reduced set of images may be operated on going forward, including being used to analyze, diagnose or treat a patient. The reduced set of images (which may be a modified version of the original set) may be combined with the 3D model and/or metadata, including comments regarding the selection process (e.g., what feature are selected, etc.); this combined 3D model and the reduced collection of images may be stored, transmitted and/or displayed.

Thus, these methods and apparatuses described herein may address well-known problems when generating intraoral scan data specific to managing the large, often extremely large, collections of data that result, which otherwise make storing, transmitting, displaying and/or analyzing too laborious and slow. Typical scan may result in hundreds of thousands of image files that may result in collections of 2D images (e.g., specific to near-IR images, color images, fluorescent images, etc.) that are generally too large to be easily transmitted and/or used. The methods and apparatuses described herein may solve this problem by implementing the novel scoring technique described herein, in particular, by combining the use of a physics-based scene classifier and one or more trained machine learning (ML) agents (e.g., an ML agent using a CNN) to score/determine which images corresponding to a 3D model give best overall coverage all potential regions of interest from a corresponding 3D model.

Thus, these methods and apparatuses (e.g., scanning systems, or systems interoperability connected to scanning systems) described herein may be used to generate one or more coverage map representations from one or more intraoral scans that may include (or may be used with) a 3D digital model of a subject's dentition, including, e.g., teeth and one or more of: gingiva palate, etc. These methods and apparatuses may generate a compact, and pre-selected coverage map that is based on the intraoral scan (or more than one intraoral scan) and that provide a highly reduced dataset of images that were taken as part of the scan. The reduced dataset may provide a coverage map that is automatically and rapidly curated, as described herein, to select for just those images that are most likely to show relevant features. These images may be displayed, stored, and/or transmitted, or may be further modified.

For example a system as described herein may be part of an intraoral scanning system that includes an intraoral scanner. The intraoral scanner may include a scanning wand including cameras and other optical components (light sources, filters, amplifiers, etc.) for scanning the patient's dentition. For example, an intraoral scanner may generally be configured to generate a plurality of two-dimensional (2D) surface images and a plurality of 2D near-infrared (near-IR) images of a subject's intraoral cavity. These images (and optionally the resulting 3D model) may be generated locally (e.g., “chairside”) and may display, transmit, and/or store the resulting images and derived structures (e.g., 3D model(s)). In some cases the apparatus may also be configured to reduce the collection of images as described herein. For example, the apparatus may include one or more processors (e.g., microprocessors, computer processors, etc.) configured to generate or receive a three-dimensional (3D) model of an intraoral 3D surface based on the plurality of surface 2D images. The one or more processors may include hardware, software and/or firmware for performing these techniques. For example, the one or more processors may identify, based on the 3D model, one or more regions from the plurality of 2D near-IR images each having one or more features of interest. The one or more regions may be identified using any of the techniques descried herein, including (but not limited to) a trained ML agent or other classifier.

The apparatus may determine a score for each of the one or more regions using a physics-based classifier and a trained neural network (e.g., a trained machine-learning agent). As described herein, the physics-based classifier may be used to score the image or one or more regions of the image, e.g., including scoring based on scene physics (“directly scoring” the images). The trained ML agent may be used in combination with and/or in parallel with the physics-based classifier.

Once scored, the method or apparatus may generate a sub-set of the 2D images selected based on the scores. In some cases a threshold may be applied, and images having a score (or an aggregate score where more than one region is scored) may be used. The threshold may be set (e.g. preset) or adjusted, including user adjusted. Alternatively or additionally the methods or apparatus may take (or reject) a predetermined number or percentage of images (e.g., the top x scoring results, where x is, e.g., 50, 100, 150, 200, 250, 300, 350, 400, 450, 500, 600, 700, 800, 900, 1000, etc., or where x is a percentage, such as the top 0.1%, 0.5%, 1%, 2%, 3%, 5%, 10%, etc.). In some cases a user may adjust the threshold or selection parameter(s). In some cases the threshold may be applied to form a sub-set of images from the initial collection (e.g., plurality) of images. In some cases the initial collection (e.g., plurality) of images may be modified directly, e.g., by removing and/or deleting 2D images (e.g., near-IR images) that are outside of the collection criterion, such as those having a score that is below a threshold from the plurality of 2D images. Thus, in general, these methods may form a reduced plurality of 2D (e.g., near-IR, color, fluorescent, etc.) images. This reduced plurality of images may be combined with the 3D model or with other sets of 2D images. For example the reduced collection of 2D images may be used as part of a reduced/compact data structure that includes the 3D model and the reduced collection of 2D images as described herein. The reduced collection of 2D images (preferably with the associated 3D model), may then be stored, transmitted, displayed, and/or analyzed, either locally (e.g., chairside) or remotely.

The resulting collection of images and associated 3D models may be referred to as a coverage map representation.

For example, FIGS. 11A-11B illustrates a visual representation of an example of a coverage map representation of an intraoral scan. The image on the left 1103 of FIG. 11A and on the left 1103′ of FIG. 11B shows 3D models of the intraoral scan over which indicator lines have been added based on the locations of corresponding 2D images (e.g., in this example, a 2D near-IR images) that have each been scored as described herein. The lines are coded to represent ranges of scores (e.g., between 0-1) corresponding to the likelihood that the image is a good representation of a particular feature or features. For example, black lines are considered worse (score 0-0.4) images, light colors are intermediate (e.g., score 0.4-0.75) images, and lightest colors represent the best/higher score images (e.g., >0.75).

In any of the methods and apparatuses described herein (including as shown in this example) the coverage map representation may use both the score, normalize or otherwise, as well as the general location on the 3D model to provide coverage over the entire 3D model (or over segmented regions or interest, e.g., teeth, gingiva, palate, etc.). For example, in FIGS. 11A-11B the images on the right 1105, 1105′ show the coverage map for the resulting reduced collection of images. In the resulting coverage mapping shown on the right side of the images 1105, 1105′, lower-scoring images (e.g., which may be indicated by more darkly shaded lines) have been removed, resulting in a much lower density of corresponding images. The resulting coverage map consists only of those images having a higher likelihood to show a feature of interest (e.g., caries, crack, wear, grinding, abscess, etc.).

Thus, in general, a coverage map may compare scores over regions of the 3D model (e.g., using a spatial window) and select those images having the highest scores (or scores above a threshold) within the region(s). These techniques may dramatically improve visualization as well as general performance of the processor, including the intraoral scanner and other post-scan processing.

In the example shown in FIG. 11, the lines illustrate camera positions where the 2D (e.g. near-IR, color, fluorescent, etc.) images were taken. The scoring alone, or in combination with the 3D model to determine which region or areas to compare against, may be used to generate a compact, reduced collection of 2D images. In the example of FIG. 11, the coverage mapping preferentially includes the high-scoring images (having score in top 25%) followed by intermediate scoring (scores in mid 40%-75%) and only includes low-scoring images (<40%) where no other higher scoring image is available.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

The present disclosure includes the following numbered clauses.

Clause 1. An intraoral scanning system, the system comprising: an intraoral scanner configured to generate a plurality of two-dimensional (2D) surface images and a plurality of 2D near-infrared (near-IR) images of a subject's intraoral cavity; and at least one processor configured to: generate or receive a three-dimensional (3D) model of an intraoral 3D surface based on the plurality of surface 2D images; identify, based on the 3D model, one or more regions from the plurality of 2D near-IR images each having one or more features of interest; determine a score for each of the one or more regions using a physics-based classifier and a trained machine learning agent; generate a reduced plurality of near-IR images based on the score from the plurality of 2D near-IR images; and store and/or transmit the reduced plurality of near-IR images.

Clause 2. The method of clause 1, wherein the at least one processor is configured to generate the reduced plurality of near-IR images based on the score and relative position of the near-IR images on the 3D model.

Clause 3. The method of clause 1, wherein the at least one processor is configured to generate the reduced plurality of near-IR images by removing or deleting images from the plurality of near-IR images to form the reduced plurality of near-IR images.

Clause 4. The system of clause 1, wherein the one or more features of interest comprises one or more of: interproximal regions, caries, cracks, wear, grinding, and/or abscesses.

Clause 5. The system of clause 1, wherein the one or more features of interest comprises caries.

Clause 6. The system of clause 1, wherein the one or more features of interest comprises interproximal regions.

Clause 7. The system of clause 1, wherein the at least one processor is further configured to segment the 3D model.

Clause 8. The system of clause 1, further comprising displaying a subset of 2D images that correspond to a predetermined range of a user-selected area from the reduced plurality of near-IR images.

Clause 9. The system of clause 8, wherein the at least one processor is further configured to receive the user-selected area from a user interface displaying the 3D model.

Clause 10. The system of clause 1, wherein using a physics-based classifier and the trained machine learning agent comprises determining the score using a physics-based scene classifier and using the trained machine learning agent focusing on local regions from the 2D images for each of the one or more regions from the plurality of 2D near-IR images.

Clause 11. The system of clause 1, wherein the trained machine learning agent is a convolutional neural network (CNN).

Clause 12. The system of clause 1, wherein the at least one processor is further configured to generate the reduced plurality of near-IR images by preserving a predetermined number or percentage images of near-IR images having the highest-ranking scores.

Clause 13. The system of clause 1, wherein the at least one processor is further configured to normalize the scores.

Clause 14. The system of clause 1, wherein the at least one processor is configured to determine the score for each of the one or more regions of each 2D near-IR image at least in part based on the physics-based classifier comprising an angle between a reference point and a camera and/or a near-IR light source corresponding to the 2D near-IR image.

Clause 15. The system of clause 1, wherein the trained machine learning agent is configured to be used in combination with one or more physics-based classifiers including one or more geometric relationship between a camera, a reference point in the one or more regions, a camera refraction, a tool surface in the one or more regions, a tooth axis, a first light source, and/or a second light source.

Clause 16. The system of clause 15, wherein the trained machine learning agent is configured to be used in combination with the physics-based classifier comprising one or more classifiers including one or more of: a cosine of an angle between a camera and a normal at a reference point, a Z absolute value of the reference point to a camera vector; a camera to reference point distance in mm; a cosine of an angle between a camera direction and the reference point; an absolute of z value of a camera direction vector; a cosine of the angle between a camera refraction and a tooth axis; an absolute z value of a light source direction vector; a cosine of an angle between a first light source and a normal; a cosine of an angle between the first light source direction and the reference point; a distance between a first light source location and the reference point in mm; a distance between a light source location and a camera in mm; a light source luminosity; an absolute of z value of the reference point to the light source vector; a cosine of an angle between a second light source direction and a normal; a second light source luminosity; an absolute of a z value of the reference point to a second light source vector; a cosine of an angle between the second light source direction and the reference point; a distance between the second light source location and the reference point in mm; and/or a distance between the second light source location and the camera in mm.

Clause 17. The system of clause 1, wherein the at least one processor is configured to store and/or transmit the 3D model with the reduced plurality of 2D near-IR images forming the reduced plurality of near-IR images.

Clause 18. An intraoral scanning system, the system comprising: an intraoral scanner configured to generate a plurality of two-dimensional (2D) surface scan images and a plurality of 2D near-infrared images; and at least one computer processor configured to: generate or receive a three-dimensional (3D) model of an intraoral 3D surface based on the plurality of surface 2D images; identify, based on the 3D model, one or more regions from the plurality of 2D near-IR images each having one or more features of interest; determine a score for each of the one or more regions using a physics-based classifier and a trained machine learning agent; generate a reduced plurality of near-IR images based on the score from the plurality of 2D near-IR images and a location of each 2D near-IR image relative to the 3D model; and store and/or transmit the reduced plurality of near-IR images.

Clause 19. A method, the method comprising: generating or receive a three-dimensional (3D) model of an intraoral 3D surface based on the plurality of surface 2D images; identifying, based on the 3D model, one or more regions from the plurality of 2D near-IR images each having one or more features of interest; determining a score for each of the one or more regions using a physics-based classifier and a trained machine learning agent; generating a reduced plurality of near-IR images based on the score from the plurality of 2D near-IR images; and storing and/or transmitting the reduced plurality of near-IR images.

Clause 20. The method of clause 19, wherein the one or more features of interest comprises one or more of: interproximal regions, caries, cracks, wear, grinding, and/or abscesses.

Clause 21. An intraoral scanning system, the system comprising: an intraoral scanner configured to: generate a plurality of scans of an intraoral three-dimensional (3D) surface of an intraoral object during intraoral scanning of the intraoral 3D surface, and generate two-dimensional (2D) near-infrared (NIR) images of the intraoral object during intraoral scanning of the intraoral 3D surface, and at least one computer processor configured to: receive the plurality of scans, build a 3D model of the intraoral 3D surface based on the plurality scans, associate at least a portion of the 2D NIR images with the 3D model such that each of the associated 2D NIR images corresponds to a position and viewing angle relative to the 3D model, display to a user the 3D model of the intraoral 3D surface, select a subset of the associated 2D NIR images that correspond to an area of the 3D model, determine a sequence for displaying the selected subset of the associated 2D NIR images, and display the selected subset of the associated 2D NIR images in the determined sequence.

Clause 22. The intraoral scanning system according to clause 21, wherein the at least one computer processor is configured to display the selected subset by projecting the selected subset of the associated 2D NIR images onto the 3D model.

Clause 23. The intraoral scanning system according to clause 21, wherein the at least one computer processor is configured to facilitate observation of caries by selecting the subset of the associated 2D NIR images based on respective viewing angles relative to the intraoral 3D surface of the associated 2D NIR images or respective NIR-illumination angles relative to the intraoral 3D surface of the associated 2D NIR images.

Clause 24. The intraoral scanning system according to clause 21, wherein the at least one computer processor is further configured to automatically identify caries in the intraoral object based on the generated 2D NIR images.

Clause 25. The intraoral scanning system according to clause 24, wherein the at least one computer processor is configured to automatically select the area of the 3D model based on the identification of caries in the intraoral object.

Clause 26. The intraoral scanning system according to clause 24, wherein the at least one computer processor is configured to select the subset of the associated 2D NIR images based on the identification of caries in the intraoral object.

Clause 27. The intraoral scanning system according to clause 21, wherein the at least one computer processor is further configured to normalize the subset of the associated 2D NIR images with respect to each other.

Clause 28. The intraoral scanning system according to clause 27, wherein the at least one computer processor is configured to normalize the subset of the associated 2D NIR images by normalizing a brightness of at least one 2D NIR image of the subset with respect to a brightness of at least one other 2D NIR image of the subset.

Clause 29. The intraoral scanning system according to clause 27, wherein the at least one computer processor is configured to normalize the subset of the associated 2D NIR images by normalizing a contrast within at least one 2D NIR image of the subset with respect to a contrast within at least one other 2D NIR image of the subset.

Clause 30. The intraoral scanning system according to clause 21, wherein the at least one computer processor is further configured to identify a common region of interest as seen in each image of the selected subset of the associated 2D NIR images, and wherein the at least one computer processor is further configured to display the selected subset in the determined sequence while maintaining the identified common region of interest spatially stable during transitioning by the computer processor from one 2D NIR image to the next within the selected subset.

Clause 31. The intraoral scanning system according to clause 30, wherein the at least one computer processor maintains the identified common region of interest spatially stable during transitioning by aligning the selected subset of the associated 2D NIR images with respect to each other.

Clause 32. The intraoral scanning system according to clause 30, wherein the at least one computer processor maintains the identified common region of interest spatially stable during transitioning by morphing at least one image of the selected subset of the associated 2D NIR images to the next.

Clause 33. The intraoral scanning system according to clause 30, wherein the at least one computer processor maintains the identified common region of interest spatially stable during transitioning by cropping at least one image of the selected subset of the associated 2D NIR images.

Clause 34. The intraoral scanning system according to clause 30, wherein: the at least one computer processor is configured to display the selected subset of the plurality of 2D NIR images in the determined sequence on a display screen, and the at least one computer processor maintains the identified common region of interest spatially stable during transitioning by displaying at least a first image of the selected subset centered around a first point on the display screen and displaying at least a second image of the selected subset centered around a second point, different from the first point, on the display screen.

Clause 35. The intraoral scanning system according to clause 34, wherein the at least one computer processor maintains the identified common region of interest spatially stable during transitioning by rotating at least one image of the selected subset of the associated 2D NIR images.

Clause 36. The intraoral scanning system according to clause 21, wherein the area of the 3D model corresponds to an interproximal area of the intraoral 3D surface.

Clause 37. The intraoral scanning system according to clause 36, wherein the at least one computer processor is further configured to: automatically identify one or more interproximal areas of the intraoral 3D surface, each identified interproximal area corresponding respectively to an area of the 3D model; for each identified interproximal area, automatically select a subset of the associated 2D NIR images that correspond to the respective area of the 3D model; and for each selected subset: determine a sequence for displaying the selected subset of the associated 2D NIR images, and display the selected subset of the associated 2D NIR images in the determined sequence.

Clause 38. The intraoral scanning system according to clause 21, wherein the at least one computer processor is further configured to: receive user input associated with a given area of the 3D model of the intraoral 3D surface, and in response to receiving the user input, select a subset of the associated 2D NIR images that correspond to the given area of the 3D model.

Clause 39. The intraoral scanning system according to clause 38, wherein the given area is an interproximal area on the 3D model corresponding to an interproximal area on the intraoral 3D surface, and the at least one computer processor is configured to receive the user input in response to the user selecting a particular one or more interproximal areas on the 3D model.

Clause 40. The intraoral scanning system according to clause 38, wherein the at least one computer processor is configured to receive the user input by the user adjusting an indicator on a view of the 3D model, wherein the given area is an area on the 3D model that is associated with the indicator.

Clause 41. The intraoral scanning system according to clause 40, wherein the indicator comprises an area-selecting indicator, wherein the user input comprises the user moving the area-selecting indicator, and wherein the given area is the area of the 3D model that is within the area-selecting indicator.

Clause 42. The intraoral scanning system according to clause 21, wherein: for one or more given viewing angles from which no 2D NIR images were generated during the intraoral scanning, the at least one computer processor is further configured to synthesize one or more synthesized 2D NIR images of the intraoral 3D surface based on 2D NIR images generated during the intraoral scanning from viewing angles nearby the one or more given viewing angles, and the computer processor is configured to display the selected subset of the associated 2D NIR images in the determined sequence by augmenting the selected subset of the associated 2D NIR images with the one or more synthesized 2D NIR images corresponding respectively to one or more viewing angles for which there is no associated 2D NIR image.

Clause 43. An intraoral scanning system, the system comprising: an intraoral scanner configured to: generate a plurality scans of an intraoral three-dimensional (3D) surface of an intraoral object during intraoral scanning of the intraoral 3D surface, and generate two-dimensional (2D) color images of the intraoral 3D surface, during intraoral scanning of the intraoral 3D surface; and at least one computer processor configured to: receive the plurality of scans, build a 3D model of the intraoral 3D surface based on the plurality of scans, associate at least a portion of the 2D color images with the 3D model such that each of the associated 2D color images corresponds to a position and viewing angle relative to the 3D model, display to a user the 3D model of the intraoral 3D surface, select a subset of the associated 2D color images that correspond to an area of the 3D model, determine a sequence for displaying the selected subset of the associated 2D color images, and display the selected subset of the associated 2D color images in the determined sequence.

Clause 44. The intraoral scanning system according to clause 43, wherein the at least one computer processor is configured to display the selected subset by displaying the selected subset of the associated 2D color images projected onto the 3D model.

Clause 45. The intraoral scanning system according to clause 43, wherein the 2D color images are images captured under white light illumination.

Clause 46. The intraoral scanning system according to clause 43, wherein the 2D color images are fluorescence images.

Clause 47. The intraoral scanning system according to clause 46, wherein the fluorescence images are images of the intraoral 3D surface fluorescing in response to ultraviolet (UV) illumination.

Clause 48. The intraoral scanning system according to clause 46, wherein the fluorescence images are images of the intraoral 3D surface fluorescing in response to red light illumination.

Clause 49. The intraoral scanning system according to clause 43, wherein the at least one computer processor is configured to facilitate observation of a dental pathology by selecting the subset of the associated 2D color images based on respective viewing angles relative to the intraoral 3D surface of the associated 2D color images.

Clause 50. The intraoral scanning system according to clause 49, wherein the dental pathology is caries.

Clause 51. The intraoral scanning system according to clause 43, wherein the at least one computer processor is further configured to automatically identify a dental pathology based on the generated 2D color images.

Clause 52. The intraoral scanning system according to clause 51, wherein the at least one computer processor is configured to automatically select the area of the 3D model based on the identification of the dental pathology.

Clause 53. The intraoral scanning system according to clause 51, wherein the at least one computer processor is configured to select the subset of the associated 2D color images based on the identification of the dental pathology.

Clause 54. The intraoral scanning system according to clause 51, wherein the dental pathology is caries.

Clause 55. The intraoral scanning system according to clause 54, wherein the at least one computer processor is configured to automatically select the area of the 3D model based on the identification of caries in the intraoral object.

Clause 56. The intraoral scanning system according to clause 54, wherein the at least one computer processor is configured to select the subset of the associated 2D color images based on the identification of caries in the intraoral object.

Clause 57. The intraoral scanning system according to clause 43, wherein the at least one computer processor is further configured to normalize the subset of the associated 2D color images with respect to each other.

Clause 58. The intraoral scanning system according to clause 57, wherein the at least one computer processor is configured to normalize the subset of the associated 2D color images by normalizing a brightness of at least one 2D color image of the subset with respect to a brightness of at least one other 2D color image of the subset.

Clause 59. The intraoral scanning system according to clause 57, wherein the at least one computer processor is configured to normalize the subset of the associated 2D color images by normalizing a contrast within at least one 2D color image of the subset with respect to a contrast within at least one other 2D color image of the subset.

Clause 60. The intraoral scanning system according to clause 43, wherein the at least one computer processor is further configured to identify a common region of interest as seen in each image of the selected subset of the associated 2D color images, and wherein the at least one computer processor is further configured to display the selected subset in the determined sequence while maintaining the identified common region of interest spatially stable during transitioning by the computer processor from one 2D color image to the next within the selected subset.

Clause 61. The intraoral scanning system according to clause 60, wherein the at least one computer processor maintains the identified common region of interest spatially stable during transitioning by aligning the selected subset of the associated 2D color images with respect to each other.

Clause 62. The intraoral scanning system according to clause 60, wherein the at least one computer processor maintains the identified common region of interest spatially stable during transitioning by morphing at least one image of the selected subset of the associated 2D color images to at least one other image of the selected subset of the associated 2D color images the next.

Clause 63. The intraoral scanning system according to clause 60, wherein the at least one computer processor maintains the identified common region of interest spatially stable during transitioning by cropping at least one image of the selected subset of the associated 2D color images.

Clause 64. The intraoral scanning system according to clause 60, wherein: the at least one computer processor is configured to display the selected subset of the plurality of 2D color images in the determined sequence on a display screen, and the at least one computer processor maintains the identified common region of interest spatially stable during transitioning by displaying at least a first image of the selected subset centered around a first point on the display screen and displaying at least a second image of the selected subset centered around a second point, different from the first point, on the display screen.

Clause 65. The intraoral scanning system according to clause 60, wherein the at least one computer processor maintains the identified common region of interest spatially stable during transitioning by rotating at least one image of the selected subset of the associated 2D color images.

Clause 66. The intraoral scanning system according to clause 43, wherein the area of the 3D model corresponds to an interproximal area of the intraoral 3D surface.

Clause 67. The intraoral scanning system according to clause 66, wherein the at least one computer processor is further configured to: automatically identify one or more interproximal areas of the intraoral 3D surface, each identified interproximal area corresponding respectively to an area of the 3D model; for each identified interproximal area, automatically select a subset of the associated 2D color images that correspond to the respective area of the 3D model; and for each selected subset: determine a sequence for displaying the selected subset of the associated 2D color images, and display the selected subset of the associated 2D color images in the determined sequence.

Clause 68. The intraoral scanning system according to clause 43, wherein the at least one computer processor is further configured to: receive user input associated with a given area of the 3D model of the intraoral 3D surface, and in response to receiving the user input, select the subset of the associated 2D color images that correspond to the given area of the 3D model.

Clause 69. The intraoral scanning system according to clause 68, wherein the given area is an interproximal area on the 3D model corresponding to an interproximal area on the intraoral 3D surface, and the at least one computer processor is configured to receive the user input in response to the user selecting a particular one or more interproximal areas on the 3D model.

Clause 70. The intraoral scanning system according to clause 68, wherein the at least one computer processor is configured to receive the user input by the user adjusting an indicator on a view of the 3D model, wherein the given area is an area on the 3D model that is associated with the indicator.

Clause 71. The intraoral scanning system according to clause 70, wherein the indicator comprises an area-selecting indicator, wherein the user input comprises the user moving the area-selecting indicator, and wherein the given area is the area of the 3D model that is within the area-selecting indicator.

Clause 72. The intraoral scanning system according to clause 43, wherein: for one or more given viewing angles from which no 2D color images were generated during the intraoral scanning, the at least one computer processor is further configured to synthesize one or more synthesized 2D color images of the intraoral 3D surface based on 2D color images generated during the intraoral scanning from viewing angles nearby the one or more given viewing angles, and the computer processor is configured to display the selected subset of the associated 2D color images in the determined sequence by augmenting the selected subset of the associated 2D color images with the one or more synthesized 2D color images corresponding respectively to one or more viewing angles for which there is no associated 2D color image.

Clause 73. An intraoral scanning system, the system comprising: (A) an intraoral scanner configured to: (i) generate a plurality of scans of an intraoral three-dimensional (3D) surface of an intraoral object during intraoral scanning of the intraoral 3D surface, and (ii) generate two-dimensional (2D) near-infrared (NIR) images of the intraoral object during intraoral scanning of the intraoral 3D surface, and (B) at least one computer processor configured to: receive the plurality of scans, build a 3D model of the intraoral 3D surface based on the plurality scans, associate at least a portion of the 2D NIR images with the 3D model such that each of the associated 2D NIR images corresponds to a position and viewing angle relative to the 3D model, display to a user the 3D model of the intraoral 3D surface, select a subset of the associated 2D NIR images that correspond to an area of the 3D model, merge the selected subset of the associated 2D NIR images into a merged 2D NIR image corresponding to the area, and display the merged 2D NIR image.

Clause 74. The intraoral scanning system according to clause 73, wherein the at least one computer processor is configured to display the merged 2D NIR image by projecting the merged 2D NIR image onto the 3D model.

Clause 75. The intraoral scanning system according to clause 73, wherein for a given area of the merged image, the at least one computer processor is configured to select specific sub-regions from each image of the selected subset to use for the given area of the merged image.

Clause 76. The intraoral scanning system according to clause 73, wherein the at least one computer processor is configured to facilitate observation of caries by selecting the subset of the associated 2D NIR images based on respective viewing angles relative to the intraoral 3D surface of the plurality of 2D NIR images or respective NIR-illumination angles relative to the intraoral 3D surface of the plurality of 2D NIR images.

Clause 77. The intraoral scanning system according to clause 73, wherein the at least one computer processor is further configured to automatically identify caries in the intraoral object based on the generated 2D NIR images.

Clause 78. The intraoral scanning system according to clause 77, wherein the at least one computer processor is configured to automatically select the area of the 3D model based on the identification of caries in the intraoral object.

Clause 79. The intraoral scanning system according to clause 77, wherein the at least one computer processor is configured to select the subset of the associated 2D NIR images based on the identification of caries in the intraoral object.

Clause 80. The intraoral scanning system according to clause 73, wherein the at least one computer processor is further configured to normalize the selected subset of the associated 2D NIR images with respect to each other and, based on the normalizing of the selected subset of the associated 2D NIR images, merge the selected subset of the associated 2D NIR images into the merged 2D NIR image.

Clause 81. The intraoral scanning system according to clause 80, wherein the at least one computer processor is configured to normalize the subset of the associated 2D NIR images by normalizing a brightness of at least one 2D NIR image of the subset with respect to a brightness of at least one other 2D NIR image of the subset.

Clause 82. The intraoral scanning system according to clause 80, wherein the at least one computer processor is configured to normalize the subset of the associated 2D NIR images by normalizing a contrast within at least one 2D NIR image of the subset with respect to a contrast within at least one other 2D NIR image of the subset.

Clause 83. The intraoral scanning system according to clause 73, wherein the at least one computer processor is further configured to identify a common region of interest as seen in each image of the subset of the associated 2D NIR images, and wherein the at least one computer processor is further configured to align the common region of interest as seen in each image of the subset of the associated 2D NIR images prior to merging the selected subset of the associated 2D NIR images into the merged 2D NIR image.

Clause 84. The intraoral scanning system according to clause 83, wherein based on the aligning of the common region of interest as seen in each image of the subset of the associated 2D NIR images prior to merging, the at least one computer processor is configured to merge the selected subset of the associated 2D NIR images by stacking the selected subset of the associated 2D NIR images into a stacked image.

Clause 85. The intraoral scanning system according to clause 84, wherein for a given area of the stacked image, the at least one computer processor is configured to select specific sub-regions from each image of the selected subset to use for the given area of the stacked image.

Clause 86. The intraoral scanning system according to clause 83, wherein the at least one computer processor aligns the identified common region of interest as seen in each image of the subset by cropping at least one image of the selected subset of the associated 2D NIR images.

Clause 87. The intraoral scanning system according to clause 83, wherein the at least one computer processor aligns the identified common region of interest as seen in each image of the subset by rotating at least one image of the selected subset of the associated 2D NIR images.

Clause 88. The intraoral scanning system according to clause 83, wherein the at least one computer processor aligns the identified common region of interest as seen in each image of the subset by morphing at least one image of the selected subset of the associated 2D NIR images to the next at least one other image of the selected subset of the associated 2D NIR images.

Clause 89. The intraoral scanning system according to clause 73, wherein the area of the 3D model corresponds to an interproximal area of the intraoral 3D surface.

Clause 90. The intraoral scanning system according to clause 89, wherein the at least one computer processor is further configured to: automatically identify one or more interproximal areas of the intraoral 3D surface, each identified interproximal area corresponding respectively to an area of the 3D model; for each identified interproximal area automatically select a subset of the associated 2D NIR images that correspond to the respective area of the 3D model; and for each selected subset: merge the selected subset of the associated 2D NIR images into a merged 2D NIR image, and display the merged 2D NIR image.

Clause 91. The intraoral scanning system according to clause 73, wherein the at least one computer processor is further configured to: receive user input associated with a given area of the 3D model of the intraoral 3D surface, and in response to receiving the user input, select the subset of the associated 2D NIR images that correspond to the given area of the 3D model.

Clause 92. The intraoral scanning system according to clause 91, wherein the given area is an interproximal area on the 3D model corresponding to an interproximal area on the intraoral 3D surface, and the at least one computer processor is configured to receive the user input in response to the user selecting a particular one or more interproximal areas on the 3D model.

Clause 93. The intraoral scanning system according to clause 91, wherein the at least one computer processor is configured to receive the user input by the user adjusting an indicator on a view of the 3D model, wherein the given area is an area on the 3D model that is associated with the indicator.

Clause 94. The intraoral scanning system according to clause 93, wherein the indicator comprises an area-selecting indicator, wherein the user input comprises the user moving the area-selecting indicator, and wherein the given area is the area of the 3D model that is within the area-selecting indicator.

Clause 95. The intraoral scanning system according to clause 73, wherein: the intraoral scanner is further configured to generate two-dimensional (2D) color images of the intraoral 3D surface, during intraoral scanning of the intraoral 3D surface, and the at least one computer processor is further configured to: associate at least a portion of the 2D color images to with the 3D model such that each of the associated 2D color images corresponds to a position and viewing angle relative to the 3D model, select a subset of the associated 2D NIR images that correspond to an area of the 3D model, select a subset of the associated 2D color images that correspond to the same area of the 3D model, merge the selected subset of the associated 2D NIR images with the selected subset of the associated 2D color images into to create a merged 2D NIR-color image corresponding to the area of the 3D model, and display the merged 2D NIR-color image.

Clause 96. An intraoral scanning system, the system comprising: (A) an intraoral scanner configured to: (i) generate a plurality scans of an intraoral three-dimensional (3D) surface of an intraoral object during intraoral scanning of the intraoral 3D surface, and (ii) generate two-dimensional (2D) color images of the intraoral 3D surface, during intraoral scanning of the intraoral 3D surface; and (B) at least one computer processor configured to: receive the plurality of scans, build a 3D model of the intraoral 3D surface based on the plurality of scans, associate at least a portion of the 2D color images with the 3D model such that each of the associated 2D color images corresponds to a position and viewing angle relative to the 3D model, display to a user the 3D model of the intraoral 3D surface, select a subset of the associated 2D color images that correspond to an area of the 3D model, merge the selected subset of the associated 2D color images into a merged 2D color image corresponding to the area, and display the merged 2D color image.

Clause 97. The intraoral scanning system according to clause 96, wherein the at least one computer processor is configured to display the merged 2D color image by projecting the merged 2D color image onto the 3D model.

Clause 98. The intraoral scanning system according to clause 96, wherein the 2D color images are images captured under white light illumination.

Clause 99. The intraoral scanning system according to clause 96, wherein the 2D color images are fluorescence images.

Clause 100. The intraoral scanning system according to clause 99, wherein the fluorescence images are images of the intraoral 3D surface fluorescing in response to ultraviolet (UV) illumination.

Clause 101. The intraoral scanning system according to clause 99, wherein the fluorescence images are images of the intraoral 3D surface fluorescing in response to red light illumination.

Clause 102. The intraoral scanning system according to clause 96, wherein for a given area of the merged image, the at least one computer processor is configured to select specific sub-regions from each image of the selected subset to use for the given area of the merged image.

Clause 103. The intraoral scanning system according to clause 96, wherein the at least one computer processor is configured to facilitate observation of a dental pathology by selecting the subset of the associated 2D color images based on respective viewing angles relative to the intraoral 3D surface of the associated 2D color images.

Clause 104. The intraoral scanning system according to clause 103, wherein the dental pathology is caries.

Clause 105. The intraoral scanning system according to clause 96, wherein the at least one computer processor is further configured to automatically identify a dental pathology based on the generated 2D color images.

Clause 106. The intraoral scanning system according to clause 104, wherein the at least one computer processor is configured to automatically select the area of the 3D model based on the identification of the dental pathology.

Clause 107. The intraoral scanning system according to clause 104, wherein the at least one computer processor is configured to select the subset of the associated 2D color images that based on the identification of the dental pathology.

Clause 108. The intraoral scanning system according to clause 104, wherein the dental pathology is caries.

Clause 109. The intraoral scanning system according to clause 108, wherein the at least one computer processor is configured to automatically select the area of the 3D model based on the identification of caries in the intraoral object.

Clause 110. The intraoral scanning system according to clause 108, wherein the at least one computer processor is configured to select the subset of the associated 2D color images that based on the identification of caries in the intraoral object.

Clause 111. The intraoral scanning system according to clause 104, wherein the at least one computer processor is further configured to normalize the selected subset of the associated 2D color images with respect to each other and based on the normalizing of the selected subset of the associated 2D color images, merge the selected subset of the associated 2D color images into the merged 2D color image.

Clause 112. The intraoral scanning system according to clause 111, wherein the at least one computer processor is configured to normalize the subset of the associated 2D color images by normalizing a brightness of at least one 2D color image of the subset with respect to a brightness of at least one other 2D color image of the subset.

Clause 113. The intraoral scanning system according to clause 111, wherein the at least one computer processor is configured to normalize the subset of the associated 2D color images by normalizing a contrast within at least one 2D color image of the subset with respect to a contrast within at least one other 2D color image of the subset.

Clause 114. The intraoral scanning system according to clause 96, wherein the at least one computer processor is further configured to identify a common region of interest as seen in each image of the subset of the associated 2D color images, and wherein the at least one computer processor is further configured to align the common region of interest as seen in each image of the subset of the associated 2D color images prior to merging the selected subset of the associated 2D color images into the merged 2D color image.

Clause 115. The intraoral scanning system according to clause 114, wherein based on the aligning of the common region of interest as seen in each image of the subset of the associated 2D color images prior to the merging, the at least one computer processor is configured to merge the selected subset of the associated 2D color images by stacking the selected subset of the associated 2D color images into a stacked image.

Clause 116. The intraoral scanning system according to clause 115, wherein for a given area of the stacked image, the at least one computer processor is configured to select specific sub-regions from each image of the selected subset to use for the given area of the stacked image.

Clause 117. The intraoral scanning system according to clause 115, wherein the at least one computer processor aligns the identified common region of interest as seen in each image of the subset by cropping at least one image of the selected subset of the associated 2D color images.

Clause 118. The intraoral scanning system according to clause 115, wherein the at least one computer processor aligns the identified common region of interest as seen in each of image of the subset by rotating at least one image of the selected subset of the associated 2D color images.

Clause 119. The intraoral scanning system according to clause 115, wherein the at least one computer processor aligns the identified common region of interest as seen in each image of the subset by morphing at least one image of the selected subset of the associated 2D color images.

Clause 120. The intraoral scanning system according to clause 96, wherein the area of the 3D model corresponds to an interproximal area of the intraoral 3D surface.

Clause 121. The intraoral scanning system according to clause 100, wherein the at least one computer processor is further configured to: automatically identify one or more interproximal areas of the intraoral 3D surface, each identified interproximal area corresponding respectively to an area of the 3D model; for each identified interproximal area, automatically select a subset of the associated 2D color images that correspond to the respective area of the 3D model; and for each selected subset: merge the selected subset of the associated 2D color images into a merged 2D color image, and display the merged 2D color image.

Clause 122. The intraoral scanning system according to clause 96, wherein the at least one computer processor is further configured to: receive user input associated with a given area of the 3D model of the intraoral 3D surface, and, in response to receiving the user input, select the subset of the associated 2D color images that correspond to the given area of the 3D model.

Clause 123. The intraoral scanning system according to clause 122, wherein the given area is an interproximal area on the 3D model corresponding to an interproximal area on the intraoral 3D surface, and the at least one computer processor is configured to receive the user input in response to the user selecting a particular one or more interproximal areas on the 3D model.

Clause 124. The intraoral scanning system according to clause 122, wherein the at least one computer processor is configured to receive the user input by the user adjusting an indicator on a view of the 3D model, wherein the given area is an area on the 3D model that is associated with the indicator.

Clause 125. The intraoral scanning system according to clause 124, wherein the indicator comprises an area-selecting indicator, wherein the user input comprises the user moving the area-selecting indicator, and wherein the given area is the area of the 3D model that is within the area-selecting indicator.

Clause 126. An intraoral scanning system, the system comprising: an intraoral scanner configured to generate a plurality of two-dimensional (2D) near-infrared images and surface 2D images; and at least one computer processor configured to: build a three-dimensional (3D) model of an intraoral 3D surface based on the plurality of surface 2D images; identify, based on the 3D model, images of the plurality of 2D near-IR images having one or more interproximal regions; determine a score for each of the one or more interproximal regions; and select a subset of 2D near-IR images from the plurality of 2D near-IR images based on the scores for regions within a predetermined range of a user-selected area; and display the subset of 2D near-IR images.

Clause 127. The system of clause 126, wherein the at least one computer processor is further configured to segment the 3D model.

Clause 128. The system of clause 126, wherein the at least one computer processor is further configured to receive the user-selected area.

Clause 129. The system of clause 126, wherein the at least one computer processor is further configured to select the user-selected area from a user interface displaying the 3D model.

Clause 130. The system of clause 126, wherein the at least one computer processor is further configured to determine the score for each of the one or more interproximal regions using a physics-based classifier and a trained machine learning agent.

Clause 131. The system of clause 130, wherein the trained machine learning agent is a convolutional machine learning agent (CNN).

Clause 132. The system of clause 126, wherein the at least one computer processor is further configured to select the subset of 2D near-IR images from the plurality of 2D near-IR images based on a predetermined number of the highest-ranking scores.

Clause 133. The system of clause 126, wherein the at least one computer processor is further configured to select the subset of 2D near-IR images from the plurality of 2D near-IR images based on comparison of the scores to a threshold value.

Clause 134. An intraoral scanning system, the system comprising: an intraoral scanner configured to generate a plurality of two-dimensional (2D) near-infrared images and surface 2D images; and at least one computer processor configured to: build a three-dimensional (3D) model of an intraoral 3D surface based on the plurality of surface 2D images; receive a user-selected area selected from a user interface displaying the 3D model; identify, based on the 3D model, images of the plurality of 2D near-IR images having one or more interproximal regions; determine a score for each of the one or more interproximal regions using a physics-based classifier and a trained machine learning agent; and select a subset of 2D near-IR images from the plurality of 2D near-IR images based on the scores for regions within a predetermined range of the user-selected area; and display the subset of 2D near-IR images.

Clause 135. A method, the method comprising: building a three-dimensional (3D) model of an intraoral 3D surface based on the plurality of surface 2D images; identifying, based on the 3D model, images of the plurality of 2D near-IR images having one or more interproximal regions; determining a score for each of the one or more interproximal regions; and selecting a subset of 2D near-IR images from the plurality of 2D near-IR images based on the scores for regions within a predetermined range of a user-selected area; and displaying the subset of 2D near-IR images.

Clause 136. The method of clause 135, further comprising segmenting the 3D model.

Clause 137. The method of clause 135, further comprising receiving the user-selected area.

Clause 138. The method of clause 135, further comprising selecting the user-selected area from a user interface displaying the 3D model.

Clause 139. The method of clause 135, further comprising determining the score for each of the one or more interproximal regions using a physics-based classifier and a trained machine learning agent.

Clause 140. The method of clause 139, wherein the trained machine learning agent is a convolutional neural network (CNN).

Clause 141. The method of clause 135, further comprising selecting the subset of 2D near-IR images from the plurality of 2D near-IR images based on a predetermined number of the highest-ranking scores.

Clause 142. The method of clause 135, further comprising selecting the subset of 2D near-IR images from the plurality of 2D near-IR images based on comparison of the scores to a threshold value.

Clause 143. A method, the method comprising: receiving a user-selected area selected from a user interface displaying the 3D model; identifying, based on the 3D model, images of the plurality of 2D near-IR images having one or more interproximal regions; determining a score for each of the one or more interproximal regions using a physics-based classifier and a trained machine learning agent; and selecting a subset of 2D near-IR images from the plurality of 2D near-IR images based on the scores for regions within a predetermined range of the user-selected area; and displaying the subset of 2D near-IR images.

What is claimed is:

1. An intraoral scanning system, the system comprising:
an intraoral scanner configured to generate a plurality of two-dimensional (2D) surface images and a plurality of 2D near-infrared (near-IR) images of a subject's intraoral cavity; and
at least one processor configured to:
generate or receive a three-dimensional (3D) model of an intraoral 3D surface based on the plurality of surface 2D images;
identify, based on the 3D model, one or more regions from the plurality of 2D near-IR images each having one or more features of interest;
determine a score for each of the one or more regions using a physics-based classifier and a trained machine learning agent;
generate a reduced plurality of near-IR images based on the score from the plurality of 2D near-IR images; and
store and/or transmit the reduced plurality of near-IR images.

2. The system of claim 1, wherein the at least one processor is configured to generate the reduced plurality of near-IR images based on the score and relative position of the near-IR images on the 3D model.

3. The system of claim 1, wherein the at least one processor is configured to generate the reduced plurality of near-IR images by removing or deleting images from the plurality of near-IR images to form the reduced plurality of near-IR images.

4. The system of claim 1, wherein the one or more features of interest comprises one or more of: interproximal regions, caries, cracks, wear, grinding, and/or abscesses.

5. The system of claim 1, wherein the one or more features of interest comprises caries.

6. The system of claim 1, wherein the one or more features of interest comprises interproximal regions.

7. The system of claim 1, wherein the at least one processor is further configured to segment the 3D model.

8. The system of claim 1, further comprising displaying a subset of 2D images that correspond to a predetermined range of a user-selected area from the reduced plurality of near-IR images.

9. The system of claim 8, wherein the at least one processor is further configured to receive the user-selected area from a user interface displaying the 3D model.

10. The system of claim 1, wherein using a physics-based classifier and the trained machine learning agent comprises determining the score using a physics-based scene classifier and using the trained machine learning agent focusing on local regions from the 2D images for each of the one or more regions from the plurality of 2D near-IR images.

11. The system of claim 1, wherein the trained machine learning agent is a convolutional neural network (CNN).

12. The system of claim 1, wherein the at least one processor is further configured to generate the reduced plurality of near-IR images by preserving a predetermined number or percentage images of near-IR images having the highest-ranking scores.

13. The system of claim 1, wherein the at least one processor is further configured to normalize the scores.

14. The system of claim 1, wherein the at least one processor is configured to determine the score for each of the one or more regions of each 2D near-IR image at least in part based on the physics-based classifier comprising an angle between a reference point and a camera and/or a near-IR light source corresponding to the 2D near-IR image.

15. The system of claim 1, wherein the trained machine learning agent is configured to be used in combination with one or more physics-based classifiers including one or more geometric relationship between a camera, a reference point in the one or more regions, a camera refraction, a tool surface in the one or more regions, a tooth axis, a first light source, and/or a second light source.

16. The system of claim 15, wherein the trained machine learning agent is configured to be used in combination with the physics-based classifier comprising one or more classifiers including one or more of: a cosine of an angle between a camera and a normal at a reference point, a Z absolute value of the reference point to a camera vector; a camera to reference point distance in mm; a cosine of an angle between a camera direction and the reference point; an absolute of z value of a camera direction vector; a cosine of the angle between a camera refraction and a tooth axis; an absolute z value of a light source direction vector; a cosine of an angle between a first light source and a normal; a cosine of an angle between the first light source direction and the reference point; a distance between a first light source location and the reference point in mm; a distance between a light source location and a camera in mm; a light source luminosity; an absolute of z value of the reference point to the light source vector; a cosine of an angle between a second light source direction and a normal; a second light source luminosity; an absolute of a z value of the reference point to a second light source vector; a cosine of an angle between the second light source direction and the reference point; a distance between the second light source location and the reference point in mm; and/or a distance between the second light source location and the camera in mm.

17. The system of claim 1, wherein the at least one processor is configured to store and/or transmit the 3D model with the reduced plurality of 2D near-IR images forming the reduced plurality of near-IR images.

18. An intraoral scanning system, the system comprising:
an intraoral scanner configured to generate a plurality of two-dimensional (2D) surface scan images and a plurality of 2D near-infrared images; and
at least one computer processor configured to:
generate or receive a three-dimensional (3D) model of an intraoral 3D surface based on the plurality of surface 2D images;
identify, based on the 3D model, one or more regions from the plurality of 2D near-IR images each having one or more features of interest;
determine a score for each of the one or more regions using a physics-based classifier and a trained machine learning agent;
generate a reduced plurality of near-IR images based on the score from the plurality of 2D near-IR images and a location of each 2D near-IR image relative to the 3D model; and
store and/or transmit the reduced plurality of near-IR images.

19. A method, the method comprising:
generating or receive a three-dimensional (3D) model of an intraoral 3D surface based on the plurality of surface 2D images;
identifying, based on the 3D model, one or more regions from the plurality of 2D near-IR images each having one or more features of interest;
determining a score for each of the one or more regions using a physics-based classifier and a trained machine learning agent;
generating a reduced plurality of near-IR images based on the score from the plurality of 2D near-IR images; and
storing and/or transmitting the reduced plurality of near-IR images.

20. The method of claim 19, wherein the one or more features of interest comprises one or more of: interproximal regions, caries, cracks, wear, grinding, and/or abscesses.
